SEQUENCE LISTING (12 sequences)

SEQ ID NO: 1 - protein, 498 aa, Lymphocytic choriomeningitis mammarenavirus
MGQIVTMFEALPHIIDEVINIVIIVLIIITSIKAVYNFATCGILALVSFLFLAGRSCGMYGLNGPDIYKGVYQFKSVEFDMSHLNLTMPNACSANNSHHYISMGSSGLELTFTNDSILNHNFCNLTSAFNKKTFDHTLMSIVSSLHLSIRGNSNHKAVSCDFNNGITIQYNLSFSDPQSAISQCRTFRGRVLDMFRTAFGGKYMRSGWGWAGSDGKTTWCSQTSYQYLIIQNRTWENHCRYAGPFGMSRILFAQEKTKFLTRRLAGTFTWTLSDSSGVENPGGYCLTKWMILAAELKCFGNTAVAKCNVNHDEEFCDMLRLIDYNKAALSKFKQDVESALHVFKTTVNSLISDQLLMRNHLRDLMGVPYCNYSKFWYLEHAKTGETSVPKCWLVTNGSYLNETHFSDQIEQEADNMITEMLRKDYIKRQGSTPLALMDLLMFSTSAYLISIFLHLVKIPTHRHIKGGSCPKPHRLTNKGICSCGAFKVPGVKTIWKRR

SEQ ID NO: 2 - protein, 422 aa, Vesiculovirus sp.
MSVTVKRIIDNTVVVPKLPANEDPVEYPADYFRKSKEIPLYINTTKSLSDLRGYVYQGLKSGNVSIIHVNSYLYGALKDIRGKLDKDWSSFGINIGKAGDTIGIFDLVSLKALDGVLPDGVSDASRTSADDKWLPLYLLGLYRVGRTQMPEYRKKLMDGLTNQCKMINEQFEPLVPEGRDIFDVWGNDSNYTKIVAAVDMFFHMFKKHECASFRYGTIVSRFKDCAALATFGHLCKITGMSTEDVTTWILNREVADEMVQMMLPGQEIDKADSYMPYLIDFGLSSKSPYSSVKNPAFHFWGQLTALLLRSTRARNARQPDDIEYTSLTTAGLLYAYAVGSSADLAQQFCVGDNKYTPDDSTGGLTTNAPPQGRDVVEWLGWFEDQNRKPTPDMMQYAKRAVMSLQGLREKTIGKYAKSEFDK

SEQ ID NO: 3 - protein, 265 aa, Vesiculovirus sp.
MDNLTKVREYLKSYSRLDQAVGEIDEIEAQRAEKSNYELFQEDGVEEHTKPSYFQAADDSDTESEPEIEDNQGLYAPDPEAEQVEGFIQGPLDDYADEEVDVVFTSDWKQPELESDEHGKTLRLTSPEGLSGEQKSQWLSTIKAVVQSAKYWNLAECTFEASGEGVIMKERQITPDVYKVTPVMNTHPSQSEAVSDVWSLSKTSMTFQPKKASLQPLTISLDELFSSRGEFISVGGDGRMSHKEAILLGLRYKKLYNQARVKYSL

SEQ ID NO: 4 - protein, 1955 aa, Vesiculovirus sp.
MEVHDFETDEFNDFNEDDYATREFLNPDERMTYLNHADYNLNSPLISDDIDNLIRKFNSLPIPSMWDSKNWDGVLEMLTSCQANPIPTSQMHKWMGSWLMSDNHDASQGYSFLHEVDKEAEITFDVVETFIRGWGNKPIEYIKKERWTDSFKILAYLCQKFLDLHKLTLILNAVSEVELLNLARTFKGKVRRSSHGTNICRIRVPSLGPTFISEGWAYFKKLDILMDRNFLLMVKDVIIGRMQTVLSMVCRIDNLFSEQDIFSLLNIYRIGDKIVERQGNFSYDLIKMVEPICNLKLMKLARESRPLVPQFPHFENHIKTSVDEGAKIDRGIRFLHDQIMSVKTVDLTLVIYGSFRHWGHPFIDYYTGLEKLHSQVTMKKDIDVSYAKALASDLARIVLFQQFNDHKKWFVNGDLLPHDHPFKSHVKENTWPTAAQVQDFGDKWHELPLIKCFEIPDLLDPSIIYSDKSHSMNRSEVLKHVRMNPNTPIPSKKVLQTMLDTKATNWKEFLKEIDEKGLDDDDLIIGLKGKERELKLAGRFFSLMSWKLREYFVITEYLIKTHFVPMFKGLTMADDLTAVIKKMLDSSSGQGLKSYEAICIANHIDYEKWNNHQRKLSNGPVFRVMGQFLGYPSLIERTHEFFEKSLIYYNGRPDLMRVHNNTLINSTSQRVCWQGQEGGLEGLRQKGWSILNLLVIQREAKIRNTAVKVLAQGDNQVICTQYKTKKSRNVVELQGALNQMVSNNEKIMTAIKIGTGKLGLLINDDETMQSADYLNYGKIPIFRGVIRGLETKRWSRVTCVTNDQIPTCANIMSSVSTNALTVAHFAENPINAMIQYNYFGTFARLLLMMHDPALRQSLYEVQDKIPGLHSSTFKYAMLYLDPSIGGVSGMSLSRFLIRAFPDPVTESLSFWRFIHVHARSEHLKEMSAVFGNPEIAKFRITHIDKLVEDPTSLNIAMGMSPANLLKTEVKKCLIESRQTIRNQVIKDATIYLYHEEDRLRSFLWSINPLFPRFLSEFKSGTFLGVADGLISLFQNSRTIRNSFKKKYHRELDDLIVRSEVSSLTHLGKLHLRRGSCKMWTCSATHADTLRYKSWGRTVIGTTVPHPLEMLGPQHRKETPCAPCNTSGFNYVSVHCPDGIHDVFSSRGPLPAYLGSKTSESTSILQPWERESKVPLIKRATRLRDAISWFVEPDSKLAMTILSNIHSLTGEEWTKRQHGFKRTGSALHRFSTSRMSHGGFASQSTAALTRLMATTDTMRDLGDQNFDFLFQATLLYAQITTTVARDGWITSCTDHYHIACKSCLRPIEEITLDSSMDYTPPDVSHVLKTWRNGEGSWGQEIKQIYPLEGNWKNLAPAEQSYQVGRCIGFLYGDLAYRKSTHAEDSSLFPLSIQGRIRGRGFLKGLLDGLMRASCCQVIHRRSLAHLKRPANAVYGGLIYLIDKLSVSPPFLSLTRSGPIRDELETIPHKIPTSYPTSNRDMGVIVRNYFKYQCRLIEKGKYRSHYSQLWLFSDVLSIDFIGPFSISTTLLQILYKPFLSGKDKNELRELANLSSLLRSGEGWEDIHVKFFTKDILLCPEEIRHACKFGIAKDNNKDMSYPPWGRESRGTITTIPVYYTTTPYPKMLEMPPRIQNPLLSGIRLGQLPTGAHYKIRSILHGMGIHYRDFLSCGDGSGGMTAALLRENVHSRGIFNSLLELSGSVMRGASPEPPSALETLGGDKSRCVNGETCWEYPSDLCDPRTWDYFLRLKAGLGLQIDLIVMDMEVRDSSTSLKIETNVRNYVHRILDEQGVLIYKTYGTYICESEKNAVTILGPMFKTVDLVQTEFSSSQTSEVYMVCKGLKKLIDEPNPDWSSINESWKNLYAFQSSEQEFARAKKVSTYFTLTGIPSQFIPDPFVNIETMLQIFGVPTGVSHAAALKSSDRPADLLTISLFYMAIISYYNINHIRVGPIPPNP

SEQ ID NO: 5 - protein, 229 aa, Vesiculovirus sp.
MSSLKKILGLKGKGKKSKKLGIAPPPYEEDTSMEYAPSAPIDKSYFGVDEMDTYDPNQLRYEKFFFTVKMTVRSNRPFRTYSDVAAAVSHWDHMYIGMAGKRPFYKILAFLGSSNLKATPAVLADQGQPEYHAHCEGRAYLPHRMGKTPPMLNVPEHFRRPFNIGLYKGTIELTMTIYDDESLEAAPMIWDHFNSSKFSDFREKALMFGLIVEKKASGAWVLDSIGHFK

SEQ ID NO: 6 - protein, 498 aa, Unknown
  Description of Unknown Dandenong virus sequence
MGQLITMFEALPHIIDEVINIVIIVLVIITSIKAVYNFATCGIIALISFCLLAGRSCGLYGVTGPDIYKGLYQFKSVEFNMSQLNLTMPNACSANNSHHYISMGKSGLELTFTNDSIISHNFCNLTDGFKKKTFDHTLMSIVASLHLSIRGNTNYKAVSCDFNNGITIQYNLSFSDAQSAINQCRTFRGRVLDMFRTAFGGKYMRSGYGWKGSDGKTTWCSQTSYQYLIIQNRTWENHCEYAGPFGLSRVLFAQEKTKFLTRRLAGTFTWTLSDSSGTENPGGYCLTKWMLIAAELKCFGNTAVAKCNINHDEEFCDMLRLIDYNKAALKKFKEDVESALHLFKTTVNSLISDQLLMRNHLRDLMGVPYCNYSKFWYLEHVKTGDTSVPKCWLVSNGSYLNETHFSDQIEQEADNMITEMLRKDYIKRQGSTPLALMDLLMFSTSAYLISVFLHLMKIPTHRHIKGGTCPKPHRLTSKGICSCGAFKVPGVKTVWKRR

SEQ ID NO: 7 - protein, 489 aa, Unknown
  Description of Unknown Dandenong virus sequence
MGQIVTFFQEVPHILEEVMNIVLMTLSILAILKGIYNVMTCGIIGLITFLFLCGRSCSSIYKDNYEFFSLDLDMSSLNATMPLSCSKNNSHHYIQVGNETGLELTLTNTSIIDHKFCNLSDAHRRNLYDKALMSILTTFHLSIPDFNQYEAMSCDFNGGKISIQYNLSHSNYVDAGNHCGTIANGIMDVFRRMYWSTSLSVASDISGTQCIQTDYKYLIIQNTSWEDHCMFSRPSPMGFLSLLSQRTRNFYISRRLLGLFTWTLSDSEGNDMPGGYCLTRSMLIGLDLKCFGNTAIAKCNQAHDEEFCDMLRLFDFNKQAISKLRSEVQQSINLINKAVNALINDQLVMRNHLRDLMGIPYCNYSKFWYLNDTRTGRTSLPKCWLVTNGSYLNETQFSTEIEQEANNMFTDMLRKEYEKRQSTTPLGLVDLFVFSTSFYLISVFLHLIKIPTHRHIKGKPCPKPHRLNHMAICSCGFYKQPGLPTQWKR

SEQ ID NO: 8 - protein, 467 aa, Homo sapiens [sequence omitted]

SEQ ID NO: 9 - protein, 433 aa, Homo sapiens
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 98 aa, Homo sapiens
MGWSCIILFLVATATGVHSSDGGAQDCCLKYSQRKIPAKVVRSYRKQEPSLGCSIPAILFLPRKRSQAELCADPKELWVQQLMQHLDKTPSPQKPAQG

SEQ ID NO: 11 - protein, 79 aa, Homo sapiens
SDGGAQDCCLKYSQRKIPAKVVRSYRKQEPSLGCSIPAILFLPRKRSQAELCADPKELWVQQLMQHLDKTPSPQKPAQG

SEQ ID NO: 12 - RNA, 11169 nt, Vesiculovirus sp.
ugcuucuguuuguuugguaauaauaguaauuuuccgaguccucuuugaaauugucauuaguuuuacagacaaugucaguucucuuaguaacuguugugucagcaucaagguuuugaaggacguuuacuccuaggucaccuuaugggccgucuaaugaagucuuuuaguuuccucuaaggagaaauguaguuaugauguuuuucaaacagucuagauucuccuauacagaugguuccggaguuuaggccuuuacauaguuaguauguacaguugucgaugaacauaccucguaauuuccuguaggccccauucaaccuauuucuaaccaguucaaagccuuauuuguagcccuuucguccccuauguuagccuuauaaacuggaacauaggaacuuucgggaccugccgcaugaaggucuaccucauagccuacgaaggucuuggucgcgucuacuguuuaccaacggaaacauagaugaaccgaauaugucucacccgucuuguguuuacggacuuaugucuuuuuucgaguaccuacccgacuguuuaguuacguuuuacuaguuacuugucaaacuuggagaacacggucuuccagcacuguaaaaacuacacaccccuuuacugucauuaauguguuuuuaacagcgacgucaccuguacaagaagguguacaaguuuuuuguacuuacacggagcaagucuaugccuugauaacaaaggucuaaguuucuaacacgacguaaccguuguaaaccuguggagacguuuuauuggccuuacagaugucuucuacauugcuggaccuagaacuuggcucuucaacgucuacuuuaccagguuuacuacgaagguccgguucuuuaacuguuccggcuaaguauguacggaauaaacuagcugaaaccuaacagaagauucagagguauaagaaggcaguuuuugggacggaaggugaagacccccguuaacugucgagaagacgagucuagguggucucguuccuuacgggcugucggacuacuguaacucauauguagagaaugaugucguccaaacaacaugcgaauacgucauccuaggagacggcugaaccguguugucaaaacacaaccucuauuguuuaugugaggucuacuaucauggccuccuaacugcugauuacguggcgguguuccgucucuacaccagcuuaccgagccuaccaaacuucuaguuuugucuuuuggcugaggacuauacuacgucauacgcuuuucucgucaguacagugacguuccggauucucucuucuguuaaccguucauacgauucagucuuaaacuguuuacugggauauuaagagucuaguggauaauauauaauacgauguauacuuuuuuugauugucuauaguaccuauuagaguguuuucaagcacucauagaguucaggauaagagcagaccuaguccgccauccucucuaucuacucuagcuucguguugcucgacuuuucagguuaauacucaacaagguucuccuaccucaccuucucguaugauucgggagaauaaaaguccgucgucuacuaagacugugucuuagacuuggucuuuaacuucuguuaguuccgaacauacguggucuaggucuucgacucguucaacuuccgaaauauguccccggaaaucuacugauacgucuacuccuucaccuacaacauaaaugaagccugaccuuugucggacucgaacuuagacugcucguaccuuucuggaaugccaacuguagcggucucccaaauucaccucucgucuuuagggucaccgaaagcugcuaauuucgucagcacguuucacgguuuaugaccuuagaccgucucacguguaaacuucguagcccucuuccccaguaauacuuccucgcggucuauugaggccuacauauauuccagugaggucacuacuuguguguaggcaggguuagucuucgucauagucuacaaaccagagagaguuucuguagguacugaaagguuggguucuuucguucagaagucggagagugguauaggaaccuacuuaacaagaguagaucuccucucaaguagagacagccuccacugccugcuuacagaguauuucuccgguaggacgagccggacucuauguuuuucaacauguuaguccgcucucaguuuauaagagacaucugauacuuuuuuucauugucuauagugcuagauucacaauaggguuagguaaguaguacucaaggaauuucuucuaagagccagacuuccccuuuccauucuuuagauucuuuaaucccuagcguggugggggaauacuucuccugugaucguaccucauacgaggcucgcgagguuaacuguuuaggauaaaaccucaacugcucuaccuguggauacuaggcuuaguuaauucuauacucuuuaagaagaaaugucacuuuuacugccaaucuagauuagcaggcaagucuuguaugagucuacaccgucggcgacauaggguaacccuaguguacauguagccuuaccgucccuuugcagggaagauguuuuagaaccgaaaaaacccaagaagauuagauuuccggugaggucgccauaaccgucuaguuccaguuggucucauagugcgagugacgcuuccgucccgaauaaacgguguauccuaccccuucuggggaggguacgaguuacauggucucgugaagucuucugguaaguuauauccagaaauguucccuugcuaacucgaguguuacugguagaugcuacuacucagugaccuucgucgaggauacuagacccuaguaaaguuaagaagguuuaaaagacuaaagucucucuuccggaauuacaaaccggacuaacagcucuuuuuccguagaccucgcacccaggaccugagauagccggugaaguuuacucgaucagauugaagaucgaagacuuguuaggggccaaaugagucagaggggauuaaggucggagagcuuguugauuauaggacagaaaagauagggauacuuuuuuugauugucucuagcuagacaaaugcgcagugccuagggggcccgacguccuuaagcggugguacccggucuagcacugguacaagcuccgggacgggguguaguagcugcuccacuaguuguagcacuaguagcacgaguaguaguaguggucguaguuccggcacauguugaagcgguggacgccguaggaccgggaccacucgaaggacaaggaccggccgucuucgacgccguacaugccggacuuaccggggcuauagauguucccgcacauggucaaguucucgcaccucaagcuguacucgguggacuuggagugguacggguugcggacgucgcgguuguuaucgguggugauguagucguacccgucgucgccggaccucaacuggaagugguugcugucguaggacuugguguugaagacguuggaguggucgcggaaguuguucuuuuggaagcuggugugggaguacucguagcacucgucggacguggacucguagucuccguugucguugguguuccggcacucgacgcugaaguuguugccguagugguaggucauguuggacucgaagucgcuaggagucucgcgguagucggucacgucuuggaagucuccgucucacgaccuguacaagucuuggcggaagccgccguucauguacucuucgccgaccccgacccggccgucgcugccguucugguggaccacgucggucuggucgauggucauggaguaguaggucuugucuuggacccucuuggugacgucuaugcggccuggaaagccguacucgucuuaggacaagcggguccucuuuugguucaaggagugguccucugaccggccguggaaguggaccugggacucgcugucgucgccgcaccucuugggaccgccgaugacggagugguucaccuacuaggaccggcggcucgacuucacgaagccguuguggcggcaccgguucacguugcacuuggugcugcuccucaagacgcuguacgacucugaguagcugauguuguuccggcgggacucguucaaguucguccugcaccucucgcgggacgugcacaaguucugguggcacuugucggaguagucgcuggucgacgaguacucuuugguggacucucuggaguacccgcacgggaugacguugaugucguucaagaccauagaccucgugcgguucuggccgcucuggucgcacggguucacgaccgaccacugguuaccgucgauggacuugcucugggugaagucgcuggucuagcucguccuucggcuguuguacuaguggcucuacgacuccuuccugauguaguucucugucccgucguggggggaccgggaguaccuagacgaguacaagucguggucgcggauggaguagucguagaaggacguggaccacuucuaggggugggugucuguguaguucccgccgucgacgggguucggggugucugagugguuguucccguagacgucgacgccgcggaaguuccacgggccgcacuuuugguagaccuucuccucuauucgccggcgaugcuggagcucuuaauuaacgaucggucuaagaaguacaaaccugguuuaguugaacacuaugguacgaguuucuccggaguuaauauaaacucaaaaauuaaaaauacuuuuuuugauugucguuaguaccuucaggugcuaaaacucuggcugcucaaguuacuaaaguuacuucuacugauacgguguucucuuaaggacuuagggcuacucgcguacugcaugaacuuaguacgacuaauguuggacuuaagaggagauuaaucacuacuauaacuguuaaauuaguccuuuaaguuaagagaagguuaagggagcuacacccuaucauucuugacccuaccucaagaacucuacaauugcaguacaguucgguuaggguaggguuguagagucuacguauuuaccuacccuucaaccaauuacagacuauuaguacuacggucaguucccauaucaaaaaauguacuucaccuguuucuccgucuuuauuguaaacugcaccaccucuggaaguaggcgccgaccccguuguuugguuaacuuauguaguuuuuccuuucuaccugacugaguaaguuuuaagagcgaauaaacacaguuuucaaaaaccugaauguguucaacuguaauuagaauuuacgacagagacuccaccuuaacgaguugaaccgcuccugaaaguuuccguuucagucuucuucaagaguaccuugcuuguauacguccuaaucccaagggucgaacccaggaugaaaauaaagucuuccuacccgaaugaaguucuuugaacuauaagauuaccuggcuuugaaagacaauuaccaguuucuacacuaauaucccuccuacguuugccacgauagguaccauacaucuuaucuguuggacaagagucucguucuguagaagagggaagauuuauagaugucuuaaccucuauuuuaacaccucuccgucccuuuaaaaagaauacugaacuaauuuuaccaccuuggcuauacguugaacuucgacuacuuuaaucguucucuuaguuccggaaaucaggguguuaagggaguaaaacuuuuaguauaguucugaagacaacuacuuccccguuuuuaacuggcuccauauucuaaggagguacuagucuauuacucacacuuuugucaccuagagugugaccacuaaauaccuagcaagucuguaaccccaguaggaaaauaucuaauaaugugaccugaucuuuuuaauguaaggguucauugguacuucuuucuauaacuacacaguauacguuuucgugaacguucacuaaaucgagccuaacaagauaaaguugucaaguuacuaguauuuuucaccaagcacuuaccucugaacgagggaguacuaguagggaaauuuucaguacaauuucuuuuauguaccgggugucgacgaguucaaguucuaaaaccucuauuuaccguacuugaaggcgacuaauuuacaaaacuuuaugggcugaaugaucuggguagcuauuauaugagacuguuuucaguaaguuacuuauccagucuccacaacuuuguacaggcuuacuuaggcuugugaggauagggaucauuuuuccacaacgucugauacaaccuguguuuccgaugguuaaccuuucuuaaagaauuucucuaacuacucuucccgaaucuacuacuacuagauuaauaaccagaauuuccuuuccucucccuugacuucaaccguccaucuaaaaagagggauuacagaaccuuuaacgcucuuaugaaacauuaauggcuuauaaacuauuucugaguaaagcagggauacaaauuuccggacuguuaccgccugcuagauugacgucaguaauuuuucuacaaucuaaggaguaggccgguuccuaacuucaguauacuccguuaaacguaucgguuaguguaacuaaugcuuuuuaccuuauuggugguuuccuucaauaguuugccgggucacaaggcucaauacccggucaagaauccaauagguaggaauuagcucucuugaguacuuaaaaaacucuuuucagaauauaugauguuaccuucuggucugaacuacgcacaaguguuguugugugacuaguuaaguuggaggguugcucaaacaaccguuccuguucucccaccugaccuuccagaugccguuuuuccuaccucauaggaguuagaugaccaauaaguuucucuccgauuuuagucuuugugacgacaguuucagaaccguguuccacuauuaguucaauaaacgugugucauauuuugcuucuuuagcucuuugcaacaucuuaaugucccacgagaguuaguuuaccaaagauuauuacucuuuuaauacugacguuaguuuuaucccugucccuucaauccugaaaacuauuuacugcuacucugauacguuagacgucuaaugaacuuaauaccuuuuuauggcuaaaaggcaccucacuaaucucccaaucucugguucucuaccagugcucacugaacacagugguuacugguuuaugggugaacacgauuauauuacucgagucaaagguguuuacgagaguggcaucgaguaaaacgacucuuggguuaguuacgguacuaugucauguuaauaaaacccuguaaacgaucugagaacaacuacuacguacuaggacgagaagcaguuaguaacauacuucaaguucuauucuauggcccgaacgugucaagaugaaaguuuaugcgguacaacauaaaccugggaagguaaccuccucacagcccguacagaaacagguccaaaaacuaaucucggaagggucuagggcauugucuuucagagaguaagaccucuaaguagguacauguacgagcuucacucguagacuuccucuacucacgucauaaaccuuuggggcucuaucgguucaaagcuuauugaguguaucuguucgaucaucuucuagguuggagagacuuguagcgauacccuuacucaggucgcuugaacaauuucugacuccaauuuuuuacgaauuagcuuaguucuguuugguaguccuugguccacuaauuccuacguugguauauaaacauaguacuucuccuagccgagucuucaaagaauaccaguuauuuaggagacaagggaucuaaaaauucacuuaaguuuaguccgugaaaaaacccucagcgucugcccgaguagucagauaaaguuuuaagagcaugauaagccuugaggaaauucuuuuucauaguaucccuuaaccuacuaaacuaacacuccucacuccauaggagaaacuguguaaaucccuuugaaguaaacucuuccccuaguacauuuuacaccuguacaagucgaugaguacgacuguguaauucuauguuuaggaccccggcaugucaauaacccuguugacaugggguagguaaucuuuacaacccagguguuguagcuuuucucugaggaacacgugguacauuguguagucccaaguuaauacaaagacacguaacaggucugcccuagguacugcagaaaucaagugccccugguaacggacgaauagaucccagauuuuguagacuuagauguagauaaaacgucggaacccuuucccuuucguuucagggugacuaauuuucucgaugugcagaaucucuacgauagagaaccaaacaacuugggcugagauuugaucguuacugauaugaaagauuguaggugagaaauuguccgcuucuuaccugguuuuccgucguacccaaguuuucuugucccagacgggaaguauccaaaagcuguagagccuacucgguaccacccaagcguagagucucgugacgucguaacugguccaacuaccguugaugucugugguacucccuagacccucuagucuuaaagcugaaaaauaagguucguugcaacgagauacgaguuuaaugguggugacaacguucucugccuaccuaguggucaacaugucuaguaauaguauaacggacauucaggacaaacucuggguaucuucucuagugggaccugaguucauaccugaugugcgggggucuacauaggguacacgacuucuguaccuccuuaccccuuccaagcaccccuguucucuauuuugucuagauaggaaaucuucccuuaaccuucuuaaaucguggacgacucguuaggauaguucagccgucuacauauccaaaagauauaccucugaaccgcauaucuuuuagaugaguacggcuccugucaagagauaaaggagauagauauguuccagcauaaucuccagcuccaaagaauuuucccaacgaucugccuaauuacucucguucaacgacgguucauuauguggccucuucagaccgaguaaacuucuccggccgguugcgucacaugccuccaaacuaaaugaacuaacuauuuaacucacauaguggagguaaggaaagagaaugaucuaguccuggauaaucucugcuuaaucuuugcuaagggguguucuaggguuggaggauaggcuguucguuggcacuauacccccacuaacagucuuuaaugaaguuuaugguuacggcagauuaacuuuucccuuuuaugucuaguguaauaaguguuaauaccaauaagagucuacagaauagguaucugaaguaaccugguaagagauaaagguggugggagaacguuuaggauauguucgguaaaaauagacccuuucuauucuuacucaacucucucgaccguuuagaaagaaguaacgauucuaguccucuccccacccuucuguauguacacuuuaagaagugguuccuguauaauaacacaggucuccuuuagucuguacgaacguucaagcccuaacgauuccuauuauuauuucuguacucgauagggggaaccccuucccuuaggucucccuguuaauguuguuagggacaaauaauaugcugguggggaauggguuucuacgaucucuacggagguucuuagguuuuaggggacgacaggccuuaguccaacccgguuaaugguugaccgcgaguaauauuuuaagccucauauaauguaccuuacccuuagguaaugucccugaagaacucaacaccucugccgaggccucccuacugacgacguaaugaugcucuuuuacacguaucgucuccuuauaaguuaucagacaaucuuaauagucccagucaguacgcuccgcggagaggacucggggggucacgggaucuuugaaauccuccucuauuuagcucuacacauuuaccacuuuguacaacccuuauagguagacugaauacacuggguuccugaacccugauaaaggaggcugaguuucguccgaaccccgaaguuuaacuaaauuaacauuaccuauaccuucaagcccuaagaagaugaucggacuuuuaacucugcuuacaaucuuuaauacacguggccuaaaaccuacucguuccucaaaauuagauguucugaauaccuuguauauaaacacucucgcuuuucuuacgucauuguuaggaaccaggguacaaguucugccagcugaaucaaguuugucuuaaaucaucaagaguuugcagacuucauauauaccauacauuuccaaacuucuuuaauuagcuacuuggguuagggcuaaccagaagguaguuacuuaggaccuuuuuggacaugcguaaggucaguagucuuguccuuaaacggucucguuucuuccaaucauguaugaaauggaacuguccauaagggaggguuaaguaaggacuaggaaaacauuuguaacucugauacgauguuuauaagccucaugggugcccacacagaguacgccgacggaauuuuaguagacuaucuggacgucuaaauaacugguaaucggaaaaaauauaccgcuaauauagcauaauauuguaguuaguauagucucauccuggcuauggaggcuugggggguagucuaccuuaacguguuuuacaccccuagcgauauugaccauauucgaaaaccgacucaaacuaccucuuucuguaaggugauauaguugucacaaaucgucaauaggucguuaguaagggcuaauccacccuccgacaaagucauuuuccuccuauguucgucuucaccucaugaucuccacuacccgaggguuuucuaugggcuuaaagucugaggaaccgggguuagcccuugaccuagucuagagaccuuaaccaggcuuugguucaagcagauuuagguaaguuacucuagaacaaguuagucgauacagcaugucaccuauuaguaaacuuuaccaguuuaaacgcuucuuuguguccuuacuaacuuaccuaguuaucugcuuaaaguuuucuucuggccagauaugacuacaacuucucacuggaugugcuccuuuugagaaccucucuaauuuuuuaguacuccucugagguuugaaauucauacuuuuuuugaaacuaggaauucugggagaacaccaaaaauaaaaaauagaccaaaacaccagaagca

(19) United States
(12) Patent Application Publication
(10) Pub. No.: US 2022/0010286 A1
(43) Pub. Date: Jan. 13, 2022

(54) PROCESS FOR PRODUCING A PURIFIED RHABDOVIRUS FROM CELL CULTURE

(71) Applicant: Boehringer Ingelheim International GmbH, Ingelheim am Rhein (DE)

(72) Inventors: Dethardt MUELLER, Vienna (AT); Alan Pardo GARCIA, Innsbruck (AT); Tanja Anneliese GREIN, Willingshausen (DE); Friedrich KAESS, Biberach an der Riss (DE); Judy NG, Innsbruck (AT); Katharina PESTA, Brixlegg (AT); Sabrina SCHNEIDER, Innsbruck (AT); Jordan TURNBULL, København (DK)

(21) Appl. No.: 17/371,159

(22) Filed: Jul. 9, 2021

(30) Foreign Application Priority Data
Jul. 10, 2020 (EP) 20185118.5

(51) Int. Cl. C12N 7/00

(52) U.S. Cl. CPC C12N 7/00; C12N 2760/20221; C12N 2760/20252

(57) ABSTRACT

The present invention relates to the field of upstream and downstream processing and provides a process for producing a purified rhabdovirus from a cell culture, preferably a purified oncolytic rhabdovirus and in particular a vesicular stomatitis virus, including pharmaceutical compositions comprising the rhabdovirus.

### SEQUENCE LISTING

The instant application contains a Sequence Listing which has been submitted electronically in ASCII format and is hereby incorporated by reference in its entirety. Said ASCII copy, created on Jul. 8, 2021, is named 01-3429-US-1_SL.txt and is 62,485 bytes in size.

### FIELD OF THE INVENTION

The present invention relates to the field of upstream and downstream processing and provides a method and a process for preparing, producing and/or purifying a rhabdovirus from a cell culture, preferably an oncolytic rhabdovirus and in particular a vesicular stomatitis virus, including pharmaceutical compositions comprising the rhabdovirus.

### BACKGROUND OF THE INVENTION

The manufacturing process design for oncolytic viruses needs to consider several critical quality attributes of the final drug substance and drug product. Among these are the high content of infectious virus per volume, and the low acceptance levels of product- and process-related impurities. Virus content per volume in drug product may be in a range of 10⁹ to 10¹² infectious or genomic units per dose, and requires concentration of active virus by at least one order of magnitude during downstream purification. Acceptable impurity thresholds of typical biologicals may still apply, as e.g. 10 ng/dose of host cell DNA (WHO limit), and pose another critical challenge when combined with the high product content that is required. Both restrictions are typically several orders of magnitude stronger than those found in other active virus products for pharmaceutical use, e.g. viral vaccines. Therefore, manufacturing procedures for a commercial scale need to be optimized and newly developed for this given purpose. In particular, there is a need for improved methods for preparing, producing or purifying rhabdoviruses from a cell culture.

For example, WO2007/123961 discloses a purification process for isolating purified vesicular stomatitis virus from cell culture. In this process the cell culture is loaded after clarifying and filtering on an anion exchange membrane adsorber and the virus is then eluted followed by a further purification and filtering step.

### SUMMARY OF THE INVENTION

The present invention addresses the above needs by providing a method and a process to obtain purified rhabdovirus, such as a vesicular stomatitis virus, from a cell culture.

It is to be understood that any embodiment relating to a specific aspect might also be combined with another embodiment also relating to that specific aspect, even in multiple tiers and combinations comprising several embodiments to that specific aspect.

In a first aspect, the present invention relates to a method of producing a rhabdovirus in a cell culture comprising the step of:
(i) obtaining a rhabdovirus harvest from the cell culture by:
   a. adding directly to the cell culture a viral release agent, followed by clarifying the cell culture, preferably via depth filtration, tangential flow filtration or centrifugation, and recovery of the rhabdovirus harvest in the supernatant,
   OR
   b. subjecting the cell culture to a filtration step, preferably depth filtration followed by rinsing of the filter, preferably depth filter with a viral release agent, and recovery of the rhabdovirus harvest in the supernatant.

In one embodiment relating to the first aspect, the rhabdovirus is a vesiculovirus, preferably a vesicular stomatitis virus. In a further related embodiment, the glycoprotein G of the vesicular stomatitis virus is replaced by the glycoprotein GP of Lymphocyte choriomeningitis virus (LCMV).

In one embodiment relating to the first aspect or any of its embodiments, the viral release agent in step (ia) is a solid salt or an aqueous salt solution, and the viral release agent in step (ib) is an aqueous salt solution. In a further related embodiment, in step (ia) the salt concentration in the cell culture is increased by at least approximately 0.01 M, 0.05 M, 0.1 M, 0.15 M, 0.2 M, 0.25 M, 0.3 M, 0.35 M, 0.4 M, 0.45 M or 0.5 M and the aqueous salt solution in step (ib) has a concentration of at least approximately 0.01 M, 0.05 M, 0.1 M, 0.15 M, 0.2 M, 0.25 M, 0.3 M, 0.35 M, 0.4 M, 0.45 M or 0.5 M. In a further related embodiment, in step (ia) the increase in salt concentration in the cell culture and in step (ib) the concentration of the aqueous salt solution is from about 0.01 M to about 5 M, about 0.05 M to about 5 M, about 0.1 M to about 5 M, about 0.15 M to about 5 M, about 0.2 M to about 5 M, about 0.25 M to about 5 M, about 0.3 M to about 5 M, about 0.35 M to about 5 M, about 0.4 M to about 5 M, about 0.45 M to about 5 M, or about 0.5 M to about 5 M.

In one embodiment relating to the first aspect or any of its embodiments, the salt is NaCl, KCl, MgCl₂, CaCl₂), NH₄Cl, NH₄ sulfate, NH₄ acetate or NH₄ bicarbonate.

In one embodiment relating to the first aspect or any of its embodiments, the viral release agent is an amino acid, preferably a polar, acidic or basic amino acid, more preferably arginine.

In one embodiment relating to the first aspect or any of its embodiments, the viral release agent is a sulfated polysaccharide, preferably dextran sulfate.

In one embodiment relating to the first aspect or any of its embodiments, wherein in step (ia) by adding the viral release agent to the cell culture the ionic strength of the cell culture is increased, preferably by at least approximately 0.01 M, or at least approximately 0.05 M, or at least approximately 0.1 M, or at least approximately 0.15 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 5 M, or from about 0.05 M to about 5 M, or from about 0.1 M to about 5 M, or from about 0.15 M to about 5 M, or from about 0.2 M to about 5 M; and in step (ib) rinsing the filter with a viral release agent containing aqueous solution having a ionic strength of at least approximately 0.01 M, or at least approximately 0.05 M, or at least approximately 0.1 M, or at least approximately 0.15 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 5 M, or from about 0.05 M to about 5 M, or from about 0.1 M to about 5 M, or from about 0.15 M to about 5 M, or from about 0.2 M to about 5 M.

In one embodiment relating to the first aspect or any of its embodiments, the rhabdovirus is produced in a mammalian host cell, preferably a HEK293 cell. In a further related embodiment, the mammalian host cell is cultured in suspension.

In one embodiment relating to the first aspect or any of its embodiments, the method of producing a rhabdovirus in a cell culture further comprises the steps of:
(ii) (Optional) reducing the salt concentration of the harvest obtained after step (ia) or (ib), preferably by dilution, diafiltration or dialysis,
(iii) (Optional) treating the rhabdovirus harvest with a DNA degrading nuclease, preferably with benzonase or salt active nuclease,
(iv) capturing the rhabdovirus by loading the solution obtained after any of steps (i) to (iii) on a cation exchanger,
(v) elution of the rhabdovirus and recovery of the eluate,
(vi) (Optional) polishing the rhabdovirus eluate of step (vii), preferably via size exclusion, multi modal size exclusion, ion exchange and/or tangential flow filtration,
(vii) (Optional) exchanging buffer of polished rhabdovirus eluate, preferably via ultrafiltration and diafiltration or dialysis,
(viii) (Optional) filtering sterilely of rhabdovirus.

In a related embodiment, the cation exchanger is a monolith, a resin, or a membrane. In a further related embodiment, the cation exchanger is a monolith adsorber.

In one embodiment relating to the first aspect or any of its embodiments, the rhabdovirus is formulated into a pharmaceutical composition.

In a second aspect, the present invention relates to a process for purifying a rhabdovirus from a cell culture infected with the rhabdovirus, comprising the step of:
(i) obtaining a rhabdovirus harvest from the cell culture by:
   a. adding directly to the cell culture a viral release agent, followed by clarifying the cell culture, preferably via depth filtration, tangential flow filtration or centrifugation, and recovery of the rhabdovirus harvest in the supernatant,
   OR
   b. subjecting the cell culture to a filtration step, preferably depth filtration followed by rinsing of the filter, preferably depth filter with a viral release agent, and recovery of the rhabdovirus harvest in the supernatant.

In one embodiment relating to the second aspect, the rhabdovirus is a vesiculovirus, preferably a vesicular stomatitis virus. In a further related embodiment, the glycoprotein G of the vesicular stomatitis virus is replaced by the glycoprotein GP of Lymphocyte choriomeningitis virus (LCMV).

In one embodiment relating to the second aspect or any of its embodiments, the viral release agent in step (ia) is a solid salt or an aqueous salt solution, and the viral release agent in step (ib) is an aqueous salt solution. In a further related embodiment, in step (ia) the salt concentration in the cell culture is increased by at least approximately 0.01 M, 0.05 M, 0.1 M, 0.15 M, 0.2 M, 0.25 M, 0.3 M, 0.35 M, 0.4 M, 0.45 M or 0.5 M and the aqueous salt solution in step (ib) has a concentration of at least approximately 0.01 M, 0.05 M, 0.1 M, 0.15 M, 0.2 M, 0.25 M, 0.3 M, 0.35 M, 0.4 M, 0.45 M or 0.5 M. In a further related embodiment, in step (ia) the increase in salt concentration in the cell culture and in step (ib) the concentration of the aqueous salt solution is from about 0.01 M to about 5 M, about 0.05 M to about 5 M, about 0.1 M to about 5 M, about 0.15 M to about 5 M, about 0.2 M to about 5 M, about 0.25 M to about 5 M, about 0.3 M to about 5 M, about 0.35 M to about 5 M, about 0.4 M to about 5 M, about 0.45 M to about 5 M, or about 0.5 M to about 5 M.

In one embodiment relating to the second aspect or any of its embodiments, the salt is NaCl, KCl, MgCl₂, CaCl₂, NH₄Cl, NH₄ sulfate, NH₄ acetate or NH₄ bicarbonate.

In one embodiment relating to the second aspect or any of its embodiments, the viral release agent is an amino acid, preferably a polar, acidic or basic amino acid, more preferably arginine.

In one embodiment relating to the second aspect or any of its embodiments, the viral release agent is a sulfated polysaccharide, preferably dextran sulfate.

In one embodiment relating to the second aspect or any of its embodiments, wherein in step (ia) by adding the viral release agent to the cell culture the ionic strength of the cell culture is increased, preferably by at least approximately 0.01 M, or at least approximately 0.05 M, or at least approximately 0.1 M, or at least approximately 0.15 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 5 M, or from about 0.05 M to about 5 M, or from about 0.1 M to about 5 M, or from about 0.15 M to about 5 M, or from about 0.2 M to about 5 M; and in step (ib) rinsing the filter with a viral release agent containing aqueous solution having a ionic strength of at least approximately 0.01 M, or at least approximately 0.05 M, or at least approximately 0.1 M, or at least approximately 0.15 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 5 M, or from about 0.05 M to about 5 M, or from about 0.1 M to about 5 M, or from about 0.15 M to about 5 M, or from about 0.2 M to about 5 M.

In one embodiment relating to the second aspect or any of its embodiments, the rhabdovirus is produced in a mammalian host cell, preferably a HEK293 cell. In a further related embodiment, the mammalian host cell is cultured in suspension.

In one embodiment relating to the second aspect or any of its embodiments, the process for purifying a rhabdovirus from a cell culture infected with the rhabdovirus further comprises the steps of:
(ii) (Optional) reducing the salt concentration of the harvest obtained after step (ia) or (ib), preferably by dilution, diafiltration or dialysis,
(iii) (Optional) treating the rhabdovirus harvest with a DNA degrading nuclease, preferably with benzonase or salt active nuclease,
(iv) capturing the rhabdovirus by loading the solution obtained after any of steps (i) to (iii) on a cation exchanger,
(v) elution of the rhabdovirus and recovery of the eluate,
(vi) (Optional) polishing the rhabdovirus eluate of step (vii), preferably via size exclusion, multi modal size exclusion, ion exchange and/or tangential flow filtration,
(vii) (Optional) exchanging buffer of polished rhabdovirus eluate, preferably via ultrafiltration and diafiltration or dialysis,
(viii) (Optional) filtering sterilely of rhabdovirus.

In a related embodiment, the cation exchanger is a monolith, a resin, or a membrane. In a further related embodiment, the cation exchanger is a monolith adsorber.

In one embodiment relating to the second aspect or any of its embodiments, the rhabdovirus is formulated into a pharmaceutical composition.

In a third aspect the present invention relates to a vesicular stomatitis virus, wherein the glycoprotein G of the vesicular stomatitis virus is replaced by the glycoprotein GP of Lymphocyte choriomeningitis virus (LCMV), which has been produced according to the method any of the aforementioned aspects or embodiments or purified according to the process according to any of the aforementioned aspects or embodiments. In an embodiment relating to the third aspect the RNA genome of the vesicular stomatitis virus produced according to the method of any of the aforementioned aspects or embodiments or purified according to the process according to any of the aforementioned aspects or embodiments consists of a coding sequence at least 98%, at least 99% or 100% identical to SEQ ID NO: 12. In a further related embodiment to the third aspect or any of its embodiments, the amount of infectious particles is at least approximately about 1×10⁹, 2×10⁹, 3×10⁹, 4×10⁹, 5×10⁹, 6×10⁹, 7×10⁹, 8×10⁹, 9×10⁹, or at least approximately about 1×10¹⁰ as measured by TCID₅₀/mL.

### BRIEF DESCRIPTION OF THE DRAWINGS

FIG. 1: Exemplary flow chart depicting upstream and downstream process steps performed for the purification/production of a Vesicular Stomatitis Virus pseudotyped with GP of LCMV (VSV-GP).

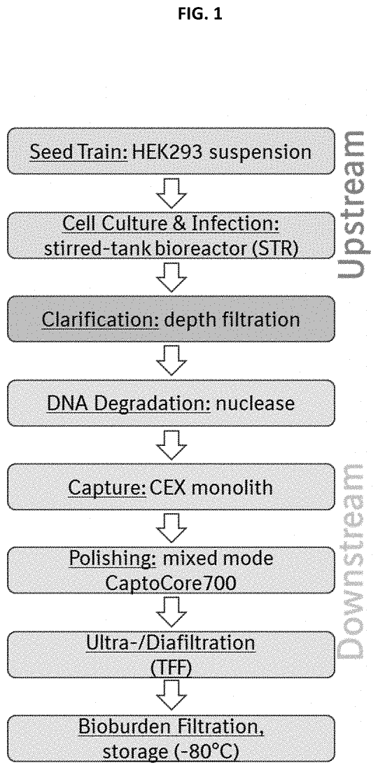

FIG. 2: Bar chart of host cell DNA purity chronologically sorted for process intermediates per dose of 1×10¹¹ TCID₅₀ VSV-GP(-Cargo) at-scale production. The upstream clarified sample (USP) was obtained by taking a sample of the crude harvest of the cell culture, adding NaCl to this crude harvest sample and then clarifying the crude harvest sample by centrifugation resulting in the USP. The host cell DNA purity was subsequently measured in the USP and host cell DNA purity was then calculated for the whole cell culture harvest. The sterile filtrated material obtained at the end of the process shows the host cell DNA purity on a drug substance level.

FIG. 3 Bar chart of host cell protein purity chronologically sorted for process intermediates per dose of 1×10¹¹ TCID₅₀ VSV-GP(-Cargo) at-scale production. The upstream clarified sample (USP) was obtained by taking a sample of the crude harvest of the cell culture, adding NaCl to this crude harvest sample and then clarifying the crude harvest sample by centrifugation resulting in the USP. The host cell protein purity was subsequently measured in the USP and host cell protein purity was then calculated for the whole cell culture harvest. The sterile filtrated material obtained at the end of the process shows the host cell protein purity on a drug substance level.

FIG. 4: Bar chart showing the TCID₅₀/mL level of VSV-GP post infection (p.i.) of HEK293F cells at the indicated time points. The TCID₅₀/mL level was determined either in the untreated crude harvest (black bars) or in the supernatant of a centrifuged sample of the treated crude harvest (grey bars). For this purpose a sample of the crude harvest was treated with different ranges of additives and then clarified by centrifugation. The TCID₅₀/mL level was determined afterwards in the supernatant of the treated crude harvest sample after centrifugation. TCID₅₀ level of crude harvest were only measured for control 30 h and 48 h p.i. and dextran sulfate treatment.

FIG. 5: Bar chart showing the genomic titer/mL level (black bars) and TCID₅₀/mL (grey bars) of VSV-GP post infection of HEK293F cells. The genomic titer/mL level or the TCID₅₀/mL level was determined in the untreated crude harvest (no additives, not centrifuged) and in the supernatant of centrifuged samples of treated crude harvest samples. For this purpose samples of crude harvest were treated with different ranges of additives and then clarified by centrifugation. The genomic titer/mL or the TCID₅₀/mL level was determined afterwards in the supernatant of the treated crude harvest samples after centrifugation.

FIG. 6: Bar chart showing the recovery of VSV-GP as measured by genomic titer/mL level (black bars) and TCID₅₀/mL (grey bars) of VSV-GP post infection of HEK293F cells. The genomic titer/mL level or the TCID₅₀/mL level was determined in the untreated crude harvest (no additives) and set at 100%. The percent recovery of VSV-GP after treatment with different concentration of salts was measured in the supernatant of centrifuged samples of treated crude harvest samples. For this purpose samples of crude harvest were treated with different ranges of salt and then clarified by centrifugation. The genomic titer/mL or the TCID₅₀/mL level was determined afterwards in the supernatant of the treated crude harvest samples after centrifugation.

FIGS. 7A-B: Representative performance data of (A) VSV-GP scalable in process control data at 50 L scale; and (B) VSV-GP scalable in process control data for 4 L. TCID₅₀/mL and genomic titer/mL levels were measured at the different unit operation steps starting with the salt treated harvest and ending with the drug substance (DS).

FIG. 8: Bar chart showing the genomic titer/mL level (black bars) and TCID₅₀/mL (grey bars) of VSV-GP post infection of HEK293F cells. The genomic titer/mL level or the TCID₅₀/mL level was determined in the supernatant of centrifuged samples of treated crude harvest samples. For this purpose samples of crude harvest were treated with different ranges of MgCl₂ or NaCl₂ and then clarified by centrifugation. The genomic titer/mL or the TCID₅₀/mL level was determined afterwards in the supernatant of the treated crude harvest samples after centrifugation.

FIG. 9: Bar chart showing the total TCID₅₀ of VSV-GP in the harvest of HEK293F infected cells. The total TCID₅₀ level was determined in the same run for the (i) crude harvest, (ii) nuclease treated crude harvest, (iii) nuclease treated crude harvest after depth filtration (clarified harvest), (iv) in the first filter flush (eluate) using tris buffer containing 0.25 M sodium chloride and (v) in the second filter flush (eluate) using tris buffer containing 0.5 M sodium chloride.

### DETAILED DESCRIPTION OF THE INVENTION

In the following detailed description, numerous specific details are set forth to provide a full understanding of the present invention. It will be apparent, however, to one ordinarily skilled in the art that the subject technology may be practiced without some of these specific details. In other instances, well-known structures and techniques have not been shown in detail so as not to obscure the present invention. The headings are included merely for convenience to assist in reading and shall not be understood to limit the invention to specific aspects or embodiments.

The inventors surprisingly found that by following the method or the process according to the invention the recovery and/or purification of rhabdovirus from cell culture is significantly improved compared to methods and processes known to the skilled artisan. The inventors have found that by adding a viral release agent and in particular a salt or dextran sulfate directly to the cell culture infected with the rhabdovirus—before the harvesting procedure is started—greatly improves the amount of rhabdovirus recoverable from the cell culture in the subsequent steps. Moreover, the recovery and/or purification of rhabdovirus is further improved by capturing the rhabdovirus on a cation exchanger. Recovered rhabdovirus may be quantitated e.g. by determining the infectious virus concentration per total TCID₅₀ or in TCID₅₀/mL by using the method of Spearman-Kärber or based on the genome copies/mL in the crude supernatant determined by qPCR.

In the broadest sense, the invention provides for a method for preparing rhabdoviruses and in particular vesicular stomatitis viruses. In one aspect, performance of the method or the process according to the invention allows for preparation, production and/or purification of rhabdovirus, preferably under cGMP conditions, resulting in high content of total infectious virus or infectious virus per volume. In a related aspect, performance of the method or the process according to the invention allows for preparation, production and/or purification of rhabdovirus sufficient for commercial purposes, i.e. to meet the demands on a commercial scale. Relating to this aspect the method/process according to the invention is preferably performed with cell cultures having a cell culture volume of at least 2 L, 5 L, 10 L, 20 L, 30 L, 40 L, 50 L, 60 L, 70 L, 80 L, 90 L, 100 L, 110 L, 120 L, 130 L, 140 L, 150 L, 160 L, 170 L, 180 L, 190 L, or at least 200 L. Further relating to this aspect, the total amount of rhabdovirus recovered may be at least approximately in the range from about 10⁸ to 10¹⁴ infectious particles measured by TCID₅₀. In particular, at least approximately about 10⁸, 10⁹, 10¹⁰, 10¹¹, 10¹², 10¹³ or 10¹⁴ infectious particles measured by TCID₅₀. Preferably, the amount of infectious particles is at least approximately about 10¹³ as measured by TCID₅₀. Further relating to this aspect, the amount of rhabdovirus recovered may be at least approximately in the range from about 10⁸ to 10¹¹ infectious particles measured by TCID₅₀/mL. In particular, at least approximately about 10⁸, 10⁹, 10¹⁰, or 10¹¹ infectious particles measured by TCID₅₀/mL. Preferably, the amount of infectious particles is at least approximately about 1×10⁹, 2×10⁹, 3×10⁹, 4×10⁹, 5×10⁹, 6×10⁹, 7×10⁹, 8×10⁹, or at least approximately about 9×10⁹, as measured by TCID₅₀/mL. More preferably, the amount of infectious particles is at least approximately about 10¹⁰ as measured by TCID₅₀/mL.

In another aspect, performance of the method or the process according to the invention will result in host cell DNA level of <10 ng/per dose, <9 ng/per dose, <8 ng/per dose, <7 ng/per dose, <6 ng/per dose, <5 ng/per dose, <4 ng/per dose, <3 ng/per dose, <2 ng/per dose, or <1 ng/per dose of host cell DNA level, wherein the dose is 1×10¹¹ total TCID₅₀ virus. Preferably, the host cell DNA level per dose is <1 ng/per dose, wherein the dose is 1×10¹¹ total TCID₅₀ virus.

In yet another aspect, performance of the method or the process according to the invention will result in host cell protein level of <10 μg/per dose, <9 μg/per dose, <8 μg/per dose, <7 μg/per dose, <6 μg/per dose, <5 μg/per dose, <4 μg/per dose, <3 μg/per dose, <2 μg/per dose, or <1 μg/per dose of host cell protein, wherein the dose is 1×10¹¹ total TCID₅₀ virus. Preferably, the host cell protein level per dose is <1 μg/per dose, wherein the dose is 1×10¹¹ total TCID₅₀ virus.

In another aspect, performance of the method or the process according to the invention will result in drug substance yields of infectious titer, as measured per total TCID₅₀, of at least 1×10¹² TCID₅₀, 2×10¹² TCID₅₀, 3×10¹² TCID₅₀, 4×10¹² TCID₅₀, 5×10¹² TCID₅₀, 6×10¹² TCID₅₀, 7×10¹² TCID₅₀, 8×10¹² TCID₅₀, 9×10¹² TCID₅₀, 1×10¹³ TCID₅₀, 1.5×10¹³ TCID₅₀, 2×10¹³ TCID₅₀, 2.5×10¹³ TCID₅₀, 3×10¹³ TCID₅₀, or 3.5×10¹³ TCID₅₀. Preferably, performance of the method or the process according to the invention in a 200 L upstream scale will result in drug substance yields of infectious titer, as measured per TCID₅₀, of at least 1×10¹³ TCID₅₀.

In a first step, a host cell is infected with the rhabdovirus, preferably with a vesicular stomatitis virus. The infection of the host cell is done by techniques routinely available to the skilled artisan and usually include inoculation of the cell culture with a rhabdovirus seed at a certain multiplicity of infection. Preferably, the infection is done at a viable cell density in the range of 1.0 to 2.0×10⁶ cells/mL. The cell culture is then cultured for a period of time to allow for sufficient rhabdovirus replication, i.e. under conditions allowing replication of the rhabdovirus. The exact conditions allowing replication of the rhabdovirus are chosen by the skilled artisan according to the specific host cell line. Preferably, the cells are infected using a Master Seed Virus at low multiplicity of infection of e.g. 0.0005 (or 5 infectious particles per 10,000 cells). Thus, the skilled artisan will understand that the method according to the invention in a first step will comprise infection of a suitable host cell with the rhabdovirus and culturing of the infected host cell under conditions allowing replication of the rhabdovirus.

The host cell may be of any origin and may be present as isolated cell or as a cell comprised in a cell population. It is preferred that the host cell producing the rhabdovirus is a mammalian cell. Alternatively, the host cell may be a human cell, monkey cell, mouse cell or hamster cell. The skilled person is aware of methods suitable for use in testing whether a given cell produces a virus and, thus, whether a particular host cell can be used within the scope of this invention. In this respect, the amount of virus produced by the host cell is not particularly limited. Preferred viral titers are >1×10⁷ TCID₅₀/mL or >1×10⁸ genome copies/mL in the crude supernatants of the given cell culture after infection without further downstream processing.

In one embodiment, the mammalian cell is a multipotent adult progenitor cell (MAPC), a neural stem cell (NSC), a mesenchymal stem cell (MSC), a HeLa cell, a HEK cell, any HEK293 cell (e.g. HEK293F or HEK293T), a Chinese hamster ovary cell (CHO), a baby hamster kidney (BHK) cell or a Vero cell or a bone marrow derived tumor infiltrating cell (BM-TIC). It is preferred to cultivate the host cell in suspension, e.g. by adapting the host cell to growth in suspension if it is not already naturally growing in suspension. In a preferred embodiment the host cell is either a HEK293F or HEK293T cell originating from a human embryonic kidney (HEK) 293 cell line. Preferably, the HEK293F/HEK293T cells are cultivated in suspension batch mode within stirred-tank or wave bioreactor systems under animal component-free and chemically defined conditions.

Host cells in the meaning of the invention include classical packaging cells for the production of rhabdovirus from non-replicable vectors as well as producer cells for the production of rhabdovirus from vectors capable of reproduction. Packaging cells usually comprise one or more plasmids for the expression of essential genes which lack in the respective vector to be packaged and/or are necessary for the production of virus. Such cells are known to the skilled person who can select appropriate cell lines suitable for the desired purpose.

In one embodiment the host cell line is a HEK293 cell. The term “HEK293 cell or HEK293 cell line” as used herein refers to an adherent human cell line that originates from human embryonic kidney and was originally immortalized in 1973 by the integration of a 4 kbp adenoviral 5 (ad5) genome fragment including the E1A and E1B genes at chromosome 19 (Graham et al., J. Gen. Virol. (1977) 36: 59-72; Malm et al., Nature research, Scientific Reports (220) 10:18996). This cell line is for example obtainable from ATCC and DSMZ (ATCC-CRL-1573; DSMZ No: ACC305; RRID:CVCL_0045). In particular, to enable large-scale cultivation and bioproduction of therapeutic proteins or virus in bioreactors the parental HEK293 cell line have also been adapted to high-density suspension growth in serum-free medium. These include, without being limited thereto the industrially relevant suspension cell lines HEK293-F, HEK293-H and FreeStyle HEK293-F cells. FreeStyle HEK293-F cells are adapted to suspension culture in FreeStyle™ 293 Expression medium and are e.g., obtainable from ThermoFisher (R79007; RRID:CVCL_D603). HEK293-F and HEK293-H cells were prepared from a clonal selection from HEK293 cells for fast growth in serum-free medium (SFM), superior transfection efficiency and a high level of protein expression and are e.g., obtainable from ThermoFisher (HEK293-F: 11625019, RRID:CVCL_6642; HEK293-H: 11631017, RRID:CVCL_6643). The HEK293-H strain is a variant, which when grown in serum supplemented medium demonstrate better adherence in monolayer culture and ease of use for plaque assays and other anchorage dependent applications. HEK293-F and HEK-293-H are provided as adapted to Gibco® CD 293 medium. Other HEK293 cell lines, without being limited thereto are e.g., HEK293.2sus (ATCC CRL-1573.3) HEK293-SF-3F6 (ATCC CRL-12585; RRID:CVCL_4V94), Expi293F (Thermofischer A14527/A14528/100044202 (cGMP banked); RRID:CVCL_D615) and HEK293-S (Ximbio 154155; RRID:CVCL_A784). HEK293 cell lines adapted to suspension growth may also be referred to as “293 cells, SFM adapted”.

For HEK293 cell cultivation the cells are passaged in a chemically defined medium in sequential batch mode inoculum stages, until sufficient cells are obtained to inoculate e.g. stirred tank reactors. Several batch passages are performed in stirred tank before inoculation with the seed virus, while dissolved oxygen, pH and temperature are controlled from stirred tank inoculation until virus harvest. The temperature during cell mass accumulation may be different from the one used after inoculation with the seed virus and usually is around 37° C. In a preferred embodiment, the temperature is shifted after infection from 37° C. down to a range of 32° C. to 36° C. and more preferably to 34° C.

In general, the method or the process according to the invention is useful to prepare, produce or purify any rhabdovirus. In a preferred embodiment the method or the process according to the invention is used to prepare, produce or purify a vesiculovirus. Particularly preferred is the preparation, production or purification of a vesicular stomatitis virus.

The family of rhabdoviruses includes 18 genera and 134 species with negative-sense, single-stranded RNA genomes of approximately 10-16 kb (Walke et al., ICTV Virus Taxonomy Profile: Rhabdoviridae, Journal of General Virology, 99:447-448 (2018)).

Characterizing features of members of the family of rhabdoviruses include one or more of the following: A bullet-shaped or bacilliform particle 100-430 nm in length and 45-100 nm in diameter comprised of a helical nucleocapsid surrounded by a matrix layer and a lipid envelope, wherein some rhabdoviruses have non-enveloped filamentous viruses. A negative-sense, single-stranded RNA of 10.8-16.1 kb, which are mostly unsegmented. A genome encoding for at least 5 genes encoding the structural proteins nucleoprotein (N), large protein (L), phosphoprotein (P), matrix protein (M), and glycoprotein (G).

As used herein a rhabdovirus can belong to the genus of: almendravirus, curiovirus, cytorhabdovirus, dichorhavirus, ephemerovirus, Hapavirus, ledantevirus, lyssavirus, novirhabdovirus, nucleorhabdovirus, perhabdovirus, sigmavirus, sprivivirus, sripuvirus, tibrovirus, tupavirus, varicosavirus or vesiculovirus.

Within the genus mentioned herein the rhabdovirus can belong to any of the listed species. The genus of almendravirus includes: arboretum almendravirus, balsa almendravirus, Coot Bay almendravirus, Puerto Almendras almendravirus, Rio Chico almendravirus; the genus of curiovirus includes: curionopolis curiovirus, Iriri curiovirus, Itacaiunas curiovirus, Rochambeau curiovirus; the genus of cythorhabdovirus includes: Alfalfa dwarf cytorhabdovirus, Barley yellow striate mosaic cytorhabdovirus, Broccoli necrotic yellows cytorhabdovirus, Colocasia bobone disease-associated cytorhabdovirus, Festuca leaf streak cytorhabdovirus, Lettuce necrotic yellows cytorhabdovirus, Lettuce yellow mottle cytorhabdovirus, Northern cereal mosaic cytorhabdovirus, Sonchus cytorhabdovirus 1, Strawberry crinkle cytorhabdovirus, Wheat American striate mosaic cytorhabdovirus; the genus of dichorhavirus includes: Coffee ringspot dichorhavirus, Orchid fleck dichorhavirus; the genus of ephemerovirus includes: Adelaide River ephemerovirus, Berrimah ephemerovirus, Bovine fever ephemerovirus, Kimberley ephemerovirus, Koolpinyah ephemerovirus, Kotonkan ephemerovirus, Obodhiang ephemerovirus, Yata ephemerovirus; the genus of hapavirus includes: Flanders hapavirus, Gray Lodge hapavirus, Hart Park hapavirus, Joinjakaka hapavirus, Kamese hapavirus, La Joya hapavirus, Landjia hapavirus, Manitoba hapavirus, Marco hapavirus, Mosqueiro hapavirus, Mossuril hapavirus, Ngaingan hapavirus, Ord River hapavirus, Parry Creek hapavirus, Wongabel hapavirus; the genus of ledantevirus includes: Barur ledantevirus, Fikirini ledantevirus, Fukuoka ledantevirus, Kanyawara ledantevirus, Kern Canyon ledantevirus, Keuraliba ledantevirus, Kolente ledantevirus, Kumasi ledantevirus, Le Dantec ledantevirus, Mount Elgon bat ledantevirus, Nishimuro ledantevirus, Nkolbisson ledantevirus, Oita ledantevirus, Wuhan ledantevirus, Yongjia ledantevirus; the genus of lyssavirus includes: Aravan lyssavirus, Australian bat lyssavirus, Bokeloh bat lyssavirus, Duvenhage lyssavirus, European bat 1 lyssavirus, European bat 2 lyssavirus, Gannoruwa bat lyssavirus, Ikoma lyssavirus, Irkut lyssavirus, Khujand lyssavirus, Lagos bat lyssavirus, Lleida bat lyssavirus, Mokola lyssavirus, Rabies lyssavirus, Shimoni bat lyssavirus, West Caucasian bat lyssavirus; the genus of novirhabdovirus includes: Hirame novirhabdovirus, Piscine novirhabdovirus, Salmonid novirhabdovirus, Snakehead novirhabdovirus; the genus of nucleorhabdovirus includes: Datura yellow vein nucleorhabdovirus, Eggplant mottled dwarf nucleorhabdovirus, Maize fine streak nucleorhabdovirus, Maize Iranian mosaic nucleorhabdovirus, Maize mosaic nucleorhabdovirus, Potato yellow dwarf nucleorhabdovirus, Rice yellow stunt nucleorhabdovirus, Sonchus yellow net nucleorhabdovirus, Sowthistle yellow vein nucleorhabdovirus, Taro vein chlorosis nucleorhabdovirus; the genus of perhabdovirus includes: Anguillid perhabdovirus, Perch perhabdovirus, Sea trout perhabdovirus; the genus of sigmavirus includes: Drosophila affinis sigmavirus, Drosophila ananassae sigmavirus, Drosophila immigrans sigmavirus, Drosophila melanogaster sigmavirus, Drosophila obscura sigmavirus, Drosophila tristis sigmavirus, Muscina stabulans sigmavirus; the genus of sprivivirus includes: Carp sprivivirus, Pike fry sprivivirus; the genus of Sripuvirus includes: Almpiwar sripuvirus, Chaco sripuvirus, Niakha sripuvirus, Sena Madureira sripuvirus, Sripur sripuvirus; the genus of tibrovirus includes: Bas-Congo tibrovirus, Beatrice Hill tibrovirus, Coastal Plains tibrovirus, Ekpoma 1 tibrovirus, Ekpoma 2 tibrovirus, Sweetwater Branch tibrovirus, tibrogargan tibrovirus; the genus of tupavirus includes: Durham tupavirus, Klamath tupavirus, Tupaia tupavirus; the genus of varicosavirus includes: Lettuce big-vein associated varicosavirus; the genus of vesiculovirus includes: Alagoas vesiculovirus, American bat vesiculovirus, Carajas vesiculovirus, Chandipura vesiculovirus, Cocal vesiculovirus, Indiana vesiculovirus, Isfahan vesiculovirus, Jurona vesiculovirus, Malpais Spring vesiculovirus, Maraba vesiculovirus, Morreton vesiculovirus, New Jersey vesiculovirus, Perinet vesiculovirus, Piry vesiculovirus, Radi vesiculovirus, Yug Bogdanovac vesiculovirus, or Moussa virus.

Preferably, the rhabdovirus prepared, produced or purified according to the method or process of the invention is an oncolytic rhabdovirus. In this respect, oncolytic has its regular meaning known in the art and refers to the ability of a rhabdovirus to infect and lyse (break down) cancer cells but not normal cells (to any significant extend). Preferably, the oncolytic rhabdovirus is capable of replication within cancer cells. Oncolytic activity may be tested in different assay systems known to the skilled artisan (an exemplary in vitro assay is described by Muik et al., Cancer Res., 74(13), 3567-78, 2014). It is to be understood that an oncolytic rhabdovirus may infect and lyse only specific types of cancer cells. Also, the oncolytic effect may vary depending on the type of cancer cells.

In a preferred embodiment, the rhabdovirus belongs to the genus of vesiculovirus. Vesiculovirus species have been defined primarily by serological means coupled with phylogenetic analysis of the genomes. Biological characteristics such as host range and mechanisms of transmission are also used to distinguish viral species within the genus. As such, the genus of vesiculovirus form a distinct monophyletic group well-supported by Maximum Likelihood trees inferred from complete L sequences.

Viruses assigned to different species within the genus vesiculovirus may have one or more of the following characteristics: A) a minimum amino acid sequence divergence of 20% in L; B) a minimum amino acid sequence divergence of 10% in N; C) a minimum amino acid sequence divergence of 15% in G; D) can be distinguished in serological tests; and E) occupy different ecological niches as evidenced by differences in hosts and or arthropod vectors.

Preferred is the vesicular stomatitis virus (VSV). In a most preferred embodiment the method or the process of the invention is used to prepare, produce or purify a vesicular stomatitis virus, wherein the glycoprotein G of the vesicular stomatitis virus is replaced with the glycoprotein GP of the lymphocytic choriomeningitis virus (LCMV), preferably of the strain WE-HPI. Such VSV (recombinant with GP of LCMV) is for example described in the WO2010/040526 and named VSV-GP.

The glycoprotein GP of the lymphocytic choriomeningitis virus (LCMV) may be GP1 or GP2. Also included are glycoproteins from different LCMV strains. In particular, LCMV-GP can be derived from LCMV wild-type or LCMV strains LCMV-WE, LCMV-WE-HPI, LCMV-WE-HPIopt. In a preferred embodiment, the gene coding for the glycoprotein GP of the LCMV encodes for a protein with an amino acid sequence as shown in SEQ ID NO:1 or an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% sequence identity to the amino acid sequence of SEQ ID NO:1 while the functional properties of the recombinant rhabdovirus comprising a glycoprotein GP encoding an amino acid sequence as shown in SEQ ID NO:1 are maintained.

The vesicular stomatitis virus, encodes in its genome usually at least for a vesicular stomatitis virus nucleoprotein (N), large protein (L), phosphoprotein (P), matrix protein (M), and a glycoprotein (G).

In a preferred embodiment the vesicular stomatitis virus encodes in its genome at least for a vesicular stomatitis virus nucleoprotein (N) comprising an amino acid sequence as set forth in SEQ ID NO:2 or a functional variant at least 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:2, a phosphoprotein (P) comprising an amino acid sequence as set forth in SEQ ID NO:3 or a functional variant at least 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:3, a large protein (L) comprising an amino acid sequence as set forth in SEQ ID NO:4 or a functional variant at least 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:4, and a matrix protein (M) comprising an amino acid sequence as set forth in SEQ ID NO:5 or a functional variant at least 80%, 81%, 82%, 83%, 84%, 85%, 86%, 87%, 88%, 89%, 90%, 91%, 92%, 93%, 94%, 95%, 96%, 97%, 98%, 99% identical to SEQ ID NO:5.

It is understood by the skilled artisan that modifications to the vesicular stomatitis virus nucleoprotein (N), large protein (L), phosphoprotein (P), matrix protein (M), or glycoprotein (G) sequence can be made without losing the basic functions of those proteins. Such functional variants as used herein retain all or part of their basic function or activity. The protein L for example is the polymerase and has an essential function during transcription and replication of the virus. A functional variant thereof must retain at least part of this ability. A good indication for retainment of basic functionality or activity is the successful production of viruses, including these functional variants, that are still capable to replicate and infect tumor cells. Production of viruses and testing for infection and replication in tumor cells may be tested in different assay systems known to the skilled artisan (an exemplary in vitro assay is described by Muik et al., Cancer Res., 74(13), 3567-78, 2014.

In a preferred embodiment the vesicular stomatitis virus encodes in its genome at least for a vesicular stomatitis virus nucleoprotein (N), large protein (L), phosphoprotein (P), matrix protein (M), and a glycoprotein (G), wherein the large protein (L) comprises an amino acid sequence having a sequence identity >80% of SEQ ID NO:4.

In a preferred embodiment the vesicular stomatitis virus encodes in its genome at least for a vesicular stomatitis virus nucleoprotein (N), large protein (L), phosphoprotein (P), matrix protein (M), and a glycoprotein (G), wherein the nucleoprotein (N) comprises an amino acid sequence having a sequence identity >90% of SEQ ID NO:2.

In a further preferred embodiment the vesicular stomatitis virus encodes in its genome at least for a vesicular stomatitis virus nucleoprotein (N), large protein (L), phosphoprotein (P), matrix protein (M), and a glycoprotein (G), wherein the large protein (L) comprises an amino acid sequence having a sequence identity equal or greater 80% of SEQ ID NO:4 and the nucleoprotein (N) comprises an amino acid sequence having a sequence identity >90% of SEQ ID NO:2.

In another embodiment the vesicular stomatitis virus glycoprotein G is replaced with the glycoprotein of the Dandenong virus (DANDV) or Mopeia (MOPV) virus. The Dandenong virus (DANDV) is an old world arenavirus. To date, there is only a single strain known to the person skilled in the art, which comprise a glycoprotein and which may be employed. The DANDV glycoprotein comprised in the vesicular stomatitis virus has more than 6 glycosylation sites, in particular 7 glycosylation sites.

An exemplary preferred glycoprotein is that as comprised in DANDV as accessible under Genbank number EU136038. In one embodiment, the gene coding for the glycoprotein of the DNADV encodes for an amino acid sequence as shown in SEQ ID NO:6 or a sequence having at least 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO:6 while the functional properties of the vesicular stomatitis virus comprising a glycoprotein encoding an amino acid sequence as shown in SEQ ID NO:6 are maintained. The Mopeia virus (MOPV) is an old world arenavirus. There are several strains known to the person skilled in the art, which comprise a glycoprotein and which may be employed as donor of the glycoprotein comprised in the vesicular stomatitis virus. The MOPV glycoprotein comprised in the vesicular stomatitis virus has more than 6 glycosylation sites, in particular 7 glycosylation sites. An exemplary preferred glycoprotein is that as comprised in Mopeia virus as accessible under Genbank number AY772170. In one embodiment, the gene coding for glycoprotein of the MOPV encodes for an amino acid sequence as shown in SEQ ID NO:7 or a sequence having at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% sequence identity to the amino acid sequence of SEQ ID NO:7 while the functional properties of the vesicular stomatitis virus comprising a glycoprotein encoding an amino acid sequence as shown in SEQ ID NO:7 are maintained.

It is to be understood that a rhabdovirus and in particular a vesicular stomatitis virus that is prepared, produced or purified according to the method or the process of the invention may encode in its genome for further genes (recombinant rhabdovirus). These genes are often termed cargos and comprise e.g. tumor antigens, chemokines, cytokines or other immunomodulatory elements. Independent of the type of cargo additionally expressed by the rhabdovirus, such cargo expressing rhabdovirus can be prepared, produced or purified according to the method or the process of the invention. Thus, in a most preferred embodiment a vesicular stomatitis virus is prepared, produced or purified according to the method or the process of the invention, wherein the glycoprotein G of the vesicular stomatitis virus is replaced with the glycoprotein GP of the lymphocytic choriomeningitis virus (LCMV). In a further preferred embodiment a vesicular stomatitis virus is prepared, produced or purified according to the method or the process of the invention, wherein the glycoprotein G of the vesicular stomatitis virus is replaced with the glycoprotein GP of the lymphocytic choriomeningitis virus (LCMV) and the genome encodes for a further cargo, such as a tumor antigen, chemokines, cytokines or other immunomodulatory elements. Preferably, the cargo is CCL21 or a C-terminally truncated CCL21 protein (having the amino acids 1-79 of full length CCL21) characterized by deletion and/or mutation of amino acid(s) in the extended c-terminus of a CCL21 protein. By deleting and/or mutating amino acids in the extended c-terminus binding to glycosaminoglycans like heparin is thereby reduced. In a further preferred embodiment the cargo is a fusion protein comprising the extracellular domain of CD80 fused to the Fc-terminus of an IgG1 resulting in a CD80Fc fusion protein. In a most preferred embodiment the cargo is a CD80 extracellular domain Fc-fusion protein, comprising or consisting of SEQ ID NO:8 or SEQ ID NO:9. In a further preferred embodiment the cargo is a CCL21 protein, comprising or consisting of SEQ ID NO: 10 or SEQ ID NO:11. However, for the purpose of the inventive method or process the inventors have found that an additional cargo does not influence the performance of method or the process and therefore the nature of the cargo shall not be construed to limit the applicability of the invention.

Rhabdovirus encoding for further genes can be produced according to methods known to the skilled artisan and include without limitation (1) using cDNAs transfected into a cell or (2) a combination of cDNAs transfected into a helper cell, or (3) cDNAs transfected into a cell, which is further infected with a helper/minivirus providing in trans the remaining components or activities needed to produce either an infectious or non-infectious recombinant rhabdovirus. Using any of these methods (e.g., helper/minivirus, helper cell line, or cDNA transfection only), the minimum components required are a DNA molecule containing the cis-acting signals for (1) encapsidation of the genomic (or antigenomic) RNA by the rhabdovirus N protein, P protein and L protein and (2) replication of a genomic or antigenomic (replicative intermediate) RNA equivalent.

A replicating element or replicon is a strand of RNA minimally containing at the 5′ and 3′ ends the leader sequence and the trailer sequence of a rhabdovirus. In the genomic sense, the leader is at the 3′ end and the trailer is at the 5′ end. Any RNA-placed between these two replication signals will in turn be replicated. The leader and trailer regions further must contain the minimal cis-acting elements for purposes of encapsidation by the N protein and for polymerase binding which are necessary to initiate transcription and replication. For preparing recombinant rhabdovirus a minivirus containing the G gene would also contain a leader region, a trailer region and a G gene with the appropriate initiation and termination signals for producing a G protein mRNA. If the minivirus further comprises an M gene, the appropriate initiation and termination signals for producing the M protein mRNA must also present.

For any gene contained within the recombinant rhabdovirus genome, the gene would be flanked by the appropriate transcription initiation and termination signals which will allow expression of those genes and production of the protein products (Schnell et al., Journal of Virology, p. 2318-2323, 1996). To produce “non-infectious” recombinant rhabdovirus, the recombinant rhabdovirus must have the minimal replicon elements and the N, P, and L proteins and it must contain the M gene. This produces virus particles that are budded from the cell, but are non-infectious particles. To produce “infectious” particles, the virus particles must additionally comprise proteins that can mediate virus particle binding and fusion, such as through the use of an attachment protein or receptor ligand. The native receptor ligand of rhabdoviruses is the G protein.

Any cell that would permit assembly of the recombinant rhabdovirus can be used. One method to prepare infectious virus particles comprises an appropriate cell line infected with a plasmid encoding for a T7 RNA polymerase or other suitable bacteriophage polymerase such as the T3 or SP6 polymerases. The cells may then be transfected with individual cDNA containing the genes encoding the G, N, P, L and M rhabdovirus proteins. These cDNAs will provide the proteins for building a recombinant rhabdovirus particle. Cells can be transfected by any method known in the art.

Also transfected into the cell line is a “polycistronic cDNA” containing the rhabdovirus genomic RNA equivalent. If the infectious, recombinant rhabdovirus particle is intended to be lytic in an infected cell, then the genes encoding for the N, P, M and L proteins must be present as well as any heterologous nucleic acid segment. If the infectious, recombinant rhabdovirus particle is not intended to be lytic, then the gene encoding the M protein is not included in the polycistronic DNA. By “polycistronic cDNA” it is meant a cDNA comprising at least transcription units containing the genes which encode the N, P and L proteins. The recombinant rhabdovirus polycistronic DNA may also contain a gene encoding a protein variant or polypeptide fragment thereof, or a therapeutic nucleic acid or protein. Alternatively, any protein to be initially associated with the viral particle first produced or fragment thereof may be supplied in trans.

The polycistronic cDNA contemplated may contain a gene encoding a protein variant, a gene encoding a reporter, a therapeutic nucleic acid, and/or either the N-P-L genes or the N-P-L-M genes. The first step in generating a recombinant rhabdovirus is expression of an RNA that is a genomic or antigenomic equivalent from a cDNA. Then that RNA is packaged by the N protein and then replicated by the P/L proteins. The recombinant virus thus produced can be recovered. If the G protein is absent from the recombinant RNA genome, then it is typically supplied in trans. If both the G and the M proteins are absent, then both are supplied in trans. For preparing “non-infectious rhabdovirus” particles, the procedure may be the same as above, except that the polycistronic cDNA transfected into the cells would contain the N, P and L genes of the rhabdovirus only. The polycistronic cDNA of non-infectious rhabdovirus particles may additionally contain a gene encoding a protein.

After transfection of the cell culture, the whole cell culture is harvested usually between one and three days after infection. Advantageously, the method/process according to the invention allows processing of the whole cell culture for the subsequent harvest step.

In a first alternative, according to the method or the process of the invention the rhabdovirus harvest is obtained from the cell culture by adding the viral release agent, such as a salt or dextran sulfate directly to the cell culture. It has been observed that although viral concentrations as measured by TCID₅₀ or per genomic copies in the crude supernatant of the harvest suggested a yield that is sufficient to meet the demands—after harvest there was a substantial loss in viral concentration which could not be explained. It was hypothesized that this may be due to interactions of the virus either with cells, debris and/or other cell culture components and that those virus are then lost after the clarification step, e.g. depth filtration, centrifugation or TFF. Without being bound by theory it is further believed that virus particles that already budded out of the host cells may be electrostatically restricted to the cell surface. Thus by e.g. increasing the ionic strength of the cell culture with a viral release agent the virus particles may be released from the cell surface. This hypothesis was tested by adding different viral release agents to the cell culture.

In this respect the term “viral release agent” or “viral releasing agent” refers to an agent—preferably formulated into a solution—that is applied to the cell culture before harvest and thereby increases the amount of virus released into the supernatant for the subsequent harvest step (as measured by TCID₅₀). The effect and suitability of a viral release agent can be easily measured by taking e.g. a sample from the cell culture before harvest, treating the sample with the viral release agent and subsequently centrifuging the treated sample and determining the amount of virus (as measured by TCID₅₀) in the supernatant of the treated sample and compare it against an untreated sample. Preferably, a viral release agent according to the invention does not (permanently) inactivate or damage the virus, does not interfere with subsequent method/process steps and/or can be easily removed during the subsequent method/process steps. Another aspect is the cost of goods: a preferred viral release agent is cost-effective and easily sourced. In one aspect, the virus harvest (as measured by TCID₅₀) obtained from the cell culture treated with the viral release agent compared to the untreated cell culture is increased by at least the factor of approximately 5, 10, 15, 20, 25, 30, 35, 40, 45, 50, 55, 60, 65, 70, 75, 80, 85, 90, 95 or 100. Preferably, the amount of virus (as measured by TCID₅₀) recovered from a harvest treated with the viral release agent compared to the untreated harvest is an increase by at least approximately the factor 10, preferably at least approximately the factor 25, preferably at least approximately by the factor 50, preferably at least approximately by the factor 75 and more preferably at least approximately by the factor 100.

For the purpose of the invention the amount needed and/or the suitability of a viral release agent to achieve the desired effect may be either expressed by (i) the increase in ionic strength in the cell culture after adding the viral release agent to the cell culture, or may alternatively expressed by (ii) the increase in concentration of the viral release agent in the cell culture. For alternative (i) it will be understood that for the purpose of the invention the viral release agent that is added to the cell culture will then effect an appropriate increase in ionic strength in the cell culture. In this context, the term “increase in ionic strength” refers to the increase in ionic strength of the cell culture after addition of the viral release agent. For alternative (ii) the “increase in viral release agent concentration” is calculated solely on the basis of the specific viral release agent that is added to the cell culture and reference is made mutatis mutandis to below term “increasing salt concentration” which describes this in more detail for the salt as a viral release agent.

Hence, in one aspect the viral release agent is an agent, preferably in a solution, that is able to increase the ionic strength of the cell culture after it has been added to the cell culture, such as salts, amino acids or sulfated polysaccharides as described herein, and thereby promotes the release of the virus particles. The increase of ionic strength in the cell culture should be at least approximately 0.01 M or at least approximately 0.05 M. In another embodiment the increase of ionic strength in in the cell culture is from about 0.01 M to about 1.5 M, or from about 0.05 M to about 1.5 M, from about 0.1 M to about 1.5 M, from about 0.15 M to about 1.5 M, or from about 0.2 M to about 1.5 M. In another embodiment the increase of ionic strength in in the cell culture is from about 0.01 M to about 5M, from 0.05 M to about 5 M, from about 0.1 M to about 5 M, from about 0.15 M to about 5 M, or from about 0.2 M to about 1.5 M. In a preferred embodiment, the increase of ionic strength in the cell culture is at least approximately 0.1 M or more preferred at least approximately 0.2 M. Depending on the type of rhabdovirus even higher increases in ionic strength may be achieved as long as the rhabdovirus is not permanently inactivated or damaged. Thus, increases of ionic strength of up to about 2 M, 2.5 M, 3 M, 3.5 M, 4 M, 4.5 M or 5 M may be achieved in the cell culture.

It is known that the ionic strength of a solution is a measure of the concentration of ions in the solution. The ionic compounds dissolve in water, dissociate into ions and result in an ionic strength of the solution which is a function of the concentration of all ions present:

$$I = \frac{1}{2} {\sum }_{i = 1}^{n} {c}_{i} {z}_{i}^{2}$$

In this equation c, is the molar concentration of the ion i (M, mol/L), z, is the charge number of that ion, and the sum is taken over all ions n in the solution. For sodium chloride the ionic strength is equal to the concentration, but for salts such as MgSO₄ the ionic strength is four times higher so that multivalent ions contribute strongly to the ionic strength. Furthermore, it is also known how a desired ionic strength of a (salt) solution, for example a buffer, may be adjusted; the setting of the ionic strength of a (salt) solution can be done depending on the concentration and ionic potency of the agents, such as salts, present. Furthermore, a vast number of publications and patent documents exist in prior art so that a specific value or range of the ionic strength may be looked up in a handbook, a monograph or the like. Therefore, the skilled person is able to provide a (salt) solution which has the required ionic strength to effect the required increase of ionic strength in the cell culture.

Preferably, by adding the viral release agent to the cell culture the ionic strength of the cell culture is increased by at least approximately 0.01 M, or at least approximately 0.05 M, at least approximately 0.1 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 1.5 M, or from about 0.05 M to about 1.5 M, or from about 0.1 M to about 1.5 M, or from about 0.15 M to about 1.5 M, or from about 0.2 M to about 1.5 M. In some instance increases in ionic strength of up to about 2 M, 2.5 M, 3 M, 3.5 M, 4 M, 4.5 M or 5 M may be achieved in the cell culture. It will be understood that any of the aforementioned lower limits and ranges may be combined with any of the higher limits or ranges.

The term “cell culture” is to be understood to comprise the host cells, the rhabdovirus and the cell culture media. The volume and/or the concentration of the viral release agent and in particular the salt or dextran sulfate is mainly dependent on the volume of the cell culture and the final concentration or ionic strength to be achieved in the cell culture. The salt may be added to the cell culture as solid salt or as an aqueous salt solution. For the salt solution, in some instances it may be appropriate to add a highly concentrated salt solution having a small volume whereas in other instances it may be desirable to add a greater volume of a salt solution having a lower salt concentration. The salt solution may be added continuously over time or it may be added all at once to the cell culture. In one embodiment the salt solution is added within a stock solution of 4 M in a dilution of 1:20 to the culture leading to a concentration increase of additional 0.2 M NaCl directly before harvest. For the dextran sulfate, it may be as well appropriate to add a highly concentrated dextran sulfate solution having a small volume whereas in other instances it may be desirable to add a greater volume of a dextran sulfate solution having a lower concentration. The dextran sulfate solution may be added continuously over time or it may be added all at once to the cell culture. In one embodiment the dextran sulfate solution is added directly before harvest to the culture leading to a concentration in the cell culture of 100 μg/ml. However, in some instances it may be required to add a dextran sulfate with lower or higher concentrations to the cell culture.

The effect and the suitability of the viral release agent and the required concentration can be easily determined by the skilled artisan as explained beforehand.

Apparently, the viral release agent and in particular the salt or dextran sulfate in the cell culture helps to dissolve aggregates of rhabdovirus that may have formed during cultivation and also supports the release of rhabdovirus from the host cell or host cell membranes. Thus, by adding the viral release agent and in particular the salt or dextran sulfate a greater amount of recoverable rhabdovirus is released into the supernatant or released from the cells which may be captured and recovered in the subsequent steps.

It is understood that also other sulfated polysaccharides then dextran sulfate are preferred. Sulfated polysaccharides with a higher degree of sulfation may be preferred. Sulfated polysaccharides may include but are not limited to dextran sulfate, pentosan polysulfate, fucoidan, and carrageenans and their respective salts.

It was also observed that by using arginine as a viral release agent the amount of rhabdovirus released in the supernatant could be improved. Thus, in one embodiment the viral release agent is a solution containing arginine. Preferably, the arginine concentration in the cell culture or the ionic strength in the cell culture is increased by addition of the solution by at least approximately 0.05 M, 0.1 M, 0.15 M, 0.2 M, 0.25 M, 0.3 M 0.35 M, 0.4 M, 0.45 M or at least approximately 0.5 M. The use of other amino acids and their respective salts is also contemplated and in one aspect amino acids and their respective salts are useful as viral release agent, preferably polar amino acids, more preferably basic or acidic amino acids. Most preferred amino acids include but are not restricted to aspartic acid, cysteine, glutamic acid, histidine, lysine or tyrosine. Preferably, the amino acid concentration in the cell culture or the ionic strength in the cell culture is increased by addition of the amino acid by at least approximately 0.01 M, 0.05 M, 0.1 M, 0.15 M, 0.2 M, 0.25 M, 0.3 M 0.35 M, 0.4 M, 0.45 M or at least approximately 0.5 M.

For the purpose of the invention it is important that by adding the salt to the cell culture, an increase in salt concentration is achieved.

In this context, the term “increasing salt concentration” always refers to the specific salt that is added to the cell culture and whether the concentration of this specific salt is increased.

It is to be understood that the cell culture may already comprise a certain level of a salt concentration in the media. The salt that may be already comprised in the cell culture and the salt that is added to the cell culture for the harvest may be the same salt or may be a different salt. If the salt in the cell culture and the salt that is added to the cell culture are the same, the skilled artisan will understand that a sufficiently concentrated salt or salt solution must be prepared to effect an increase in salt concentration taking into account the existing salt in the media. By way of example, to effect an increase by 0.2 M NaCl in a cell culture already comprising a NaCl concentration of 0.1 M (leading to a final concentration of NaCl of 0.3 M in the cell culture), a sufficiently concentrated NaCl salt solution must be prepared taking into account the existing NaCl concentration in the media.

On the other hand if the cell culture does not comprise any salt or comprises a different salt than the one that is to be added to the cell culture, than the increase in salt concentration will be solely based on the salt that will be added to the cell culture. Hence, in both such instances (no salt or different salt), the increase in salt concentration is based only on the specific salt that is added to the cell culture, i.e. the increase in salt concentration will only be calculated on the basis of the specific salt that is added to the cell culture.

The increase in salt concentration in the cell culture should be at least approximately 0.01 M or at least approximately 0.05 M. In another embodiment the increase in salt concentration in the cell culture is from about 0.01 M to about 1.5 M, from about 0.05 M to about 1.5 M, from about 0.1 M to about 1.5 M, from about 0.15 M to about 1.5 M, or from about 0.2 M to about 1.5 M.

In a preferred embodiment, the increase in salt concentration in the cell culture is at least approximately 0.1 M or more preferred at least approximately 0.2 M. The inventors tested increases in concentration up to 1.5 M without any negative impact on the rhabdovirus. Depending on the type of rhabdovirus even higher salt concentrations may be achieved as long as the rhabdovirus is not permanently inactivated or damaged by the salt concentration. Thus, increases in salt concentrations of up to about 2 M, 2.5 M, 3 M, 3.5 M, 4 M, 4.5 M or 5 M may be achieved in the cell culture.

Suitable salts include organic as well as inorganic salts. The inorganic salt is not limited according to the present invention, any inorganic salt which is soluble in an aqueous solution and does not permanently damage the cell culture and/or the virus may be employed. The inorganic salt is for example selected from the group consisting of alkali salts or alkaline earth salts of sulfates, nitrates, phosphates, carbonates, halogenides, borates, silicates and the like. If a pharmaceutically acceptable product shall be provided the inorganic salt as well as the organic salt shall be selected from the group of pharmaceutically acceptable salts per se known. For example, pharmaceutically acceptable inorganic salts are selected from sodium salts such as sodium halides, preferably sodium chloride, sodium sulfate, sodium borate; calcium salts such as calcium halides, preferably calcium chloride, calcium sulfate, calcium borate; magnesium salts such as magnesium halides, preferably magnesium chloride, magnesium sulfate, magnesium borate, and combinations thereof as well as other pharmaceutically acceptable inorganic salts.

Further examples of pharmaceutically acceptable salts include, but are not limited to, mineral or organic acid salts of basic residues such as amines; alkali or organic salts of acidic residues such as carboxylic acids; and the like. For example, such salts include salts from benzenesulfonic acid, benzoic acid, citric acid, ethanesulfonic acid, fumaric acid, gentisic acid, hydrobromic acid, hydrochloric acid, maleic acid, malic acid, malonic acid, mandelic acid, methanesulfonic acid, 4-methyl-benzenesulfonic acid, phosphoric acid, salicylic acid, succinic acid, sulfuric acid and tartaric acid. Further pharmaceutically acceptable salts can be formed with cations from ammonia, L-arginine, calcium, 2,2′-iminobisethanol, L-lysine, magnesium, N-methyl-D-glucamine, potassium, sodium and tris(hydroxymethyl)-aminomethane.

Preferred salts include, but are not limited to NaCl, KCl, MgCl₂, CaCl₂), NH₄Cl, NH₄ sulfate, NH₄ acetate or NH₄ bicarbonate. In a most preferred embodiment a NaCl salt solution is used. The salt solution may comprise a buffer and preferably a buffer with a pH from about 5 to about 9, desirably from about 6.5 to about 8.5.

It will be understood that the pH of the cell culture after addition of the viral release agent is maintained in a range that preserves the integrity of the cells and/or more importantly of the virus for example in the range from about pH 5 to 9, or more preferably from about 6.5 to 8.5. Also, in some instances it may be desirable—dependent on the nature of the viral release agent—to adjust the pH to a certain range to influence the charge of the viral release agent and thereby promote its properties as viral release agent.

After adding the viral release agent and in particular the salt or dextran sulfate to the cell culture to achieve the desired concentration or ionic strength in the cell culture no specific hold or incubation time is foreseen. Thus, after having achieved the desired concentration or ionic strength in the cell culture the whole cell culture will be further processed in the next step. It may be desirable to stir or move the cell culture to achieve an even distribution of the viral release agent in the cell culture. Due to the technical necessities of adding the viral release agent and in particular the salt or dextran sulfate and preparing or moving the cell culture to the next process steps a certain incubation time with the viral release agent is inherently achieved. In some instances it may be desirable to incubate the cell culture after having added the viral release agent for a certain period of time. This incubation time may be 1 h, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, or 24 h.

According to the method or the process of the invention the cell culture is then subjected to a clarification step. As the rhabdovirus is secreted into the supernatant this clarification step serves to separate as much of particulate matter, i.e. cells and cell debris from the supernatant containing the rhabdovirus. Different methods are available to the skilled artisan to remove the cells from the cell culture supernatant, preferably the clarification includes one or more of the following methods: depth filtration, tangential flow filtration, and/or centrifugation. In each of those clarification methods the supernatant is separated from the cells and cell debris and the supernatant with the rhabdovirus is collected for further processing. Other methods known to the skilled artisan to separate the particulate matter from the supernatant may be applied equally in this clarification step. In a preferred embodiment, the cell culture is clarified via microfiltration by cross-flow, more preferably by using a 0.65 μm cut off hollow fiber. In another preferred embodiment, the cell culture is clarified via a depth filter. In some instances—to prevent a potential bioburden contamination—a 0.45 μm/0.2 μm filtration is connected in series to the depth filter.

In a second alternative, according to the method or the process of the invention the rhabdovirus harvest is obtained from the cell culture by first clarifying the cell culture via a filtration step, preferably depth filtration followed by rinsing of the filter, preferably depth filter with the viral release agent, in particular an aqueous salt solution or dextran sulfate. If an aqueous salt solution is used, this salt solution has a concentration or ionic strength of at least approximately 0.01 M or 0.05 M, or a concentration or an ionic strength from about 0.01 M to about 1.5 M or from about 0.05 M to about 1.5 M. The supernatant (or in this alternative the filtrate) and the rinsing solution—both containing the rhabdovirus harvest—are then recovered.

Thus, in this alternative the viral release agent, in particular a salt solution or dextran sulfate is not added directly to the cell culture but the cell culture is clarified first via a filtration step, preferably depth filtration and the filter, preferably depth filter is rinsed with the viral release agent (preferably a salt solution or dextran sulfate). The cells and cell debris are retained by the filter, preferably depth filter and the viral release agent supports primarily the release of rhabdovirus from the cells or cell membranes that are retained by the filter, preferably depth filter. Thus in this alternative, the effect of adding the viral release agent is the same as in the first alternative, i.e. a greater amount of recoverable rhabdovirus is released and collected which may be captured and recovered in the subsequent steps. It will be understood that the skilled artisan can choose from a range of different filters that would allow the removal of the cells and cell debris and will select the appropriate filter.

Also here, depending on the type of rhabdovirus even higher e.g. salt concentrations may be applied, as long as the rhabdovirus is not permanently inactivated or damaged by the salt concentration. Suitable salts include organic as well as inorganic salts. The inorganic salt is not limited according to the present invention, any inorganic salt which is soluble in an aqueous solution and does not interfere with the cell culture and/or the virus may be employed. The inorganic salt is for example selected from the group consisting of alkali salts or alkaline earth salts of sulfates, nitrates, phosphates, carbonates, halogenides, borates, silicates and the like. If a pharmaceutically acceptable product shall be provided the inorganic salt as well as the organic salt shall be selected from the group of pharmaceutically acceptable salts per se known. For example, pharmaceutically acceptable inorganic salts are selected from sodium salts such as sodium halides, preferably sodium chloride, sodium sulfate, sodium borate; calcium salts such as calcium halides, preferably calcium chloride, calcium sulfate, calcium borate; magnesium salts such as magnesium halides, preferably magnesium chloride, magnesium sulfate, magnesium borate, and combinations thereof as well as other pharmaceutically acceptable inorganic salts.

Preferred salts include again, but are not limited to NaCl, KCl, MgCl₂, CaCl₂), NH₄Cl, NH₄ sulfate, NH₄ acetate or NH₄ bicarbonate. In a most preferred embodiment a NaCl salt solution is used. The salt solution may comprise a buffer and preferably a buffer with a pH from about 5 to about 9, desirably from about 6.5 to about 8.5. For the dextran sulfate, it may be as well appropriate to add a highly concentrated dextran sulfate solution having a small volume whereas in other instances it may be desirable to add a greater volume of a dextran sulfate solution having a lower concentration. The required concentration can be easily determined by the skilled artisan as explained beforehand.

Likewise, in the second alternative arginine may be used as a viral release agent. Thus, in one embodiment the viral release agent is a solution containing arginine. Preferably, the arginine concentration of the solution or the ionic strength of the solution is at least approximately 0.01 M, 0.05 M, 0.1 M, 0.15 M, 0.2 M, 0.25 M, 0.3 M 0.35 M, 0.4 M, 0.45 M or at least approximately 0.5 M. The use of other amino acids and their respective salts is also contemplated and in one aspect amino acids are useful as viral release agent, preferably polar amino acids, more preferably basic or acidic amino acids. Most preferred amino acids include but are not restricted to aspartic acid, cysteine, glutamic acid, histidine, lysine or tyrosine. Preferably, the amino acid concentration of the solution or the ionic strength of the solution is at least approximately 0.01 M, 0.05 M, 0.1 M, 0.15 M, 0.2 M, 0.25 M, 0.3 M 0.35 M, 0.4 M, 0.45 M or at least approximately 0.5 M.

In another embodiment both the first and second alternative may be combined, i.e. the rhabdovirus harvest is obtained from the cell culture by adding a viral release agent and in particular a salt or dextran sulfate directly to the cell culture followed by depth filtration of the cell culture and subsequent rinsing of the depth filter with a viral release agent (preferably a salt solution or dextran sulfate).

Optionally, in some instances it may be necessary to reduce or remove the viral release agent from the obtained rhabdovirus harvest as to not interfere with subsequent method/process steps or with the drug substance. For example, it may be that the salt concentration interferes with subsequent capture steps, DNA degrading steps or that the salt concentration may damage or inactive the rhabdovirus if incubated for longer periods of time. Reducing the salt concentration may be achieved preferably by dilution, diafiltration and/or dialysis. In any case in each of those methods the salt concentration is reduced and the rhabdovirus harvest with the reduced salt concentration is then further processed according to the method or the process of the invention. Other methods known to the skilled artisan which are suitable to reduce the salt concentration in the rhabdovirus harvest may be applied equally.

Further optionally, in some instances it may be desirable to treat the rhabdovirus harvest with a DNA degrading nuclease such as benzonase. In a preferred embodiment, the DNA degrading nuclease is a salt active nuclease, such as SAN-HQ.

The rhabdovirus harvest that is recovered after the clarification step is then subjected to a further purification step by loading the harvest on a cation exchanger. The harvest may be loaded on the cation exchanger right after the clarification step. In some instances the recovered harvest is stored for some time, e.g. overnight and then applied on the next day to the cation exchanger. In one embodiment the material of the cation exchanger is a monolith, a resin, a fibre or a membrane. In a preferred embodiment, the cation exchanger is a monolith adsorber. It was found that by using a cation exchanger capturing of the rhabdovirus was much more efficient compared to e.g. anion exchange. Preferably, this capture and purification step is performed by monolithic cation-exchange chromatography in bind/elute mode.

In one aspect, the aforementioned purification step also serves the purpose of concentrating the rhabdovirus harvest. In this respect, the (clarified) virus harvest (as measured by TCID₅₀/mL) is concentrated by at least the factor of approximately 5, 10, 15, 20, 25, 30, 35, 40, 45, 50, 55, 60, 65, 70, 75, 80, 85, 90, 95 or 100. Preferably, the virus is concentrated (as measured by TCID₅₀/mL) by at least approximately the factor 10, preferably at least approximately the factor 25, preferably at least approximately by the factor 50, preferably at least approximately by the factor 75 and more preferably at least approximately by the factor 100. By way of example, concentration of a 10 L solution of virus having a concentration of 1×10⁹ TCID₅₀/mL by a factor of 10 would result in a 1 L solution of virus with a concentration of 1×10¹⁰ TCID₅₀/mL of virus.

The buffers chosen for applying the rhabdovirus harvest on the cation exchanger are in general well known to the skilled artisan. Conditions are chosen that will result in a maximum binding of the rhabdovirus to the cation exchanger. Preferably, the harvest is diluted with a conditioning buffer before applying it to the cation exchanger. In one embodiment the conditioning buffer comprises a Tris buffer, more preferably the conditioning buffer comprises 100 mM Tris buffer adjusted to pH 7.5 with HCl. Preferably, the harvest and the conditioning buffer are diluted in a ratio of at least 1:1 or at least 1:2 before the application to the cation exchanger.

The captured rhabdovirus is then eluted from the cation exchanger. Elution is typically done by applying elution buffers to the cation exchanger with linear rising salt concentrations or by using a single step elution process. In a preferred embodiment, the elution buffer further comprises arginine.

The so purified eluate is then collected and pooled. This rhabdovirus eluate is already highly purified but depending on the intended further use of the rhabdovirus eluate one or more of the following optional processing steps may be performed with the rhabdovirus eluate:

(i) Polishing the rhabdovirus eluate to remove further impurities, preferably via size exclusion, multi modal size exclusion, ion exchange and/or tangential flow filtration.

In a preferred embodiment, the polishing step is based on a mixed-mode bead-based size exclusion and anion exchange chromatography (Capto™ Core) in flow-through mode, e.g. with an approximate exclusion limit of 700 kD.

(ii) Buffer change of the rhabdovirus eluate or the polished rhabdovirus, preferably via ultrafiltration and diafiltration or dialysis.

In a preferred embodiment, the buffer exchange is performed using Ultra-/Diafiltration followed by a bioburden filtration resulting in the Drug Substance.

(iii) Sterile filtering of the rhabdovirus.

### Pharmaceutical Compositions

To be used in therapy, the rhabdovirus prepared, produced and/or purified according to the method or the process of the invention is formulated into pharmaceutical compositions appropriate to facilitate administration to animals or humans. Typical formulations can be prepared by mixing the rhabdovirus with physiologically acceptable carriers, excipients or stabilizers, in the form of aqueous solutions or aqueous or non-aqueous suspensions. Carriers, excipients, modifiers or stabilizers are nontoxic at the dosages and concentrations employed. They include buffer systems such as phosphate, citrate, acetate and other inorganic or organic acids and their salts; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g., Zn-protein complexes); and/or ionic or non-ionic surfactants such as TWEEN™ (polysorbates), PLURONICS™ or fatty acid esters, fatty acid ethers or sugar esters. The excipients may also have a release-modifying or absorption-modifying function.

In one embodiment the rhabdovirus is formulated into a pharmaceutical composition comprising Tris, arginine and optionally citrate. Tris is preferably used in a concentration of about 1 mM to about 100 mM. Arginine is preferably used in a concentration of about 1 mM to about 100 mM. Citrate may be present in a concentration up to 100 mM. A preferred formulation comprises about 50 mM Tris and 50 mM arginine.

The pharmaceutical composition may be provided as a liquid, a frozen liquid or in a lyophilized form. The frozen liquid may be stored at temperatures between about 0° C. and about −85° C. including temperatures between −70° C. and −85° C. and of about −15° C., −16° C., −17° C., −18° C., −19° C., −20° C., −21° C., −22° C., −23° C., −24° C. or about −25° C.

The rhabdovirus or pharmaceutical composition need not be, but is optionally, formulated with one or more agents currently used to prevent or treat the disorder in question. The effective amount of such other agents depends on the amount of recombinant antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the rhabodvirus or pharmaceutical composition (when used alone or in combination with one or more other additional therapeutic agents) will depend on the type of disease to be treated, the type of rhabdovirus, the severity and course of the disease, whether the rhabdovirus is administered for preventive or therapeutic purposes, previous therapy, the patient's clinical history and response to the rhabdovirus, and the discretion of the attending physician. The rhabdovirus or pharmaceutical composition of the invention suitably administered to the patient at one time or over a series of treatments.

Depending on the type and severity of the disease, about 10⁸ to 10¹³ infectious particles measured by TCID₅₀ of the rhabdovirus can be an initial candidate dosage for administration to the patient, whether, for example, by one or more separate administrations, or by continuous infusion. For repeated administrations over several days or longer, depending on the condition, the treatment would generally be sustained until a desired suppression of disease symptoms occurs. One exemplary dosage of the rhabdovirus would be in the range from about 10⁸ to 10¹³ infectious particles measured by TCID₅₀. Thus, one or more doses of about 10⁸, 10⁹, 10¹⁰, 10¹¹, 10¹², or 10¹³ infectious particles measured by TCID₅₀ (or any combination thereof) may be administered to the patient. Such doses may be administered intermittently, e.g. every week or every three weeks (e.g. such that the patient receives from about two to about twenty, or e.g. about six doses of the recombinant rhabdovirus). An initial higher loading dose, followed by one or more lower doses or vice versa may be administered. However, other dosage regimens may be useful. The progress of this therapy is easily monitored by conventional techniques and assays.

The efficacy of the rhabdovirus and of compositions comprising the same can be tested using any suitable in vitro assay, cell-based assay, in vivo assay and/or animal model known per se, or any combination thereof, depending on the specific disease involved. Suitable assays and animal models will be clear to the skilled person, and for example include the assays and animal models used in the Examples below.

The actual pharmaceutically effective amount or therapeutic dosage will of course depend on factors known by those skilled in the art such as age and weight of the patient, route of administration and severity of disease. In any case the rhabdovirus will be administered at dosages and in a manner which allows a pharmaceutically effective amount to be delivered based upon patient's unique condition.

Alternatively, the rhabdovirus or pharmaceutical composition of the invention may be delivered in a volume of from about 50 μL to about 100 mL including all numbers within the range, depending on the size of the area to be treated, the viral titer used, the route of administration, and the desired effect of the method.

For intratumoral administration the volume is preferably between about 50 μL to about 5 mL including volumes of about 100 μL, 200 μL, 300 μL, 400 μL, 500 μL, 600 μL, 700 μL, 800 μL, 900 μL, 1000 μL, 1100 μL, 1200 μL, 1300 μL, 1400 μL, 1500 μL, 1600 μL, 1700 μL, 1800 μL, 1900 μL, 2000 μL, 2500 μL, 3000 μL, 3500 μL, 4000 μL, or about 4500 μL. In a preferred embodiment the volume is about 1000 μL.

For systemic administration, e.g. by infusion of the rhabdovirus the volumes may be naturally higher. Alternatively, a concentrated solution of the rhabdovirus could be diluted in a larger volume of infusion solution directly before infusion.

In particular for intravenous administration the volume is preferably between 1 mL and 100 mL including volumes of about 2 mL, 3 mL, 4 mL, 5 mL, 6 mL, 7 mL, 8 mL, 9 mL, 10 mL, 11 mL, 12 mL, 13 mL, 14 mL, 15 mL, 16 mL, 17 mL, 18 mL, 19 mL, 20 mL, 25 mL, 30 mL, 35 mL, 40 mL, 45 mL, 50 mL, 55 mL, 60 mL, 70 mL, 75 mL, 80 mL, 85 mL, 90 mL, 95 mL, or about 100 mL. In a preferred embodiment the volume is between about 5 mL and 15 mL, more preferably the volume is about 6 mL, 7 mL, 8 mL, 9 mL, 10 mL, 11 mL, 12 mL, 13 mL, or about 14 mL.

Preferably the same formulation is used for intratumoral administration and intravenous administration. The doses and/or volume ratio between intratumoral and intravenous administration may be about 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19 or about 1:20. For example, a doses and/or volume ratio of 1:1 means that the same doses and/or volume is administered intratumorally as well as intravenously, whereas e.g. a doses and/or volume ratio of about 1:20 means an intravenous administration dose and/or volume that is twenty times higher than the intratumoral administration dose and/or volume. Preferably, the doses and/or volume ratio between intratumoral and intravenous administration is about 1:9.

An effective concentration of a rhabdovirus desirably ranges between about 10⁸ and 10¹⁴ vector genomes per milliliter (vg/mL). The infectious units may be measured as described in McLaughlin et al., J Virol.; 62(6):1963-73 (1988). Preferably, the concentration is from about 1.5×10⁹ to about 1.5×10¹³, and more preferably from about 1.5×10⁹ to about 1.5×10¹¹. In one embodiment, the effective concentration is about 1.5×10⁹. In another embodiment, the effective concentration is about 1.5×10¹⁰. In another embodiment, the effective concentration is about 1.5×10¹¹. In yet another embodiment, the effective concentration is about 1.5×10¹². In another embodiment, the effective concentration is about 1.5×10¹³. In another embodiment, the effective concentration is about 1.5×10¹⁴. It may be desirable to use the lowest effective concentration in order to reduce the risk of undesirable effects. Still other dosages in these ranges may be selected by the attending physician, taking into account the physical state of the subject, preferably human, being treated, the age of the subject, the particular type of cancer and the degree to which the cancer, if progressive, has developed.

An effective target concentration of a rhabdovirus may be expressed with the TCID₅₀. The TCID₅₀ can be determined for example by using the method of Spearman-Kärber. Desirably ranges include an effective target concentration between 1×10⁸/mL and 1×10¹⁴/mL TCID₅₀. Preferably, the effective target concentration is from about 1×10⁹ to about 1×10¹²/mL, and more preferably from about 1×10⁹ to about 1×10¹¹/mL. In one embodiment, the effective target concentration is about 1×10¹⁰/mL. In a preferred embodiment the target concentration is 5×10¹⁰/mL. In another embodiment, the effective target concentration is about 1.5×10¹¹/mL. In one embodiment, the effective target concentration is about 1×10¹²/mL. In another embodiment, the effective target concentration is about 1.5×10¹³/mL.

An effective target dose of a rhabdovirus may also be expressed with the TCID₅₀. Desirably ranges include a target dose between 1×10⁸ and 1×10¹⁴ TCID₅₀. Preferably, the target dose is from about 1×10⁹ to about 1×10¹³, and more preferably from about 1×10⁹ to about 1×10¹². In one embodiment, the effective concentration is about 1×10¹⁰. In a preferred embodiment, the effective concentration is about 1×10¹¹. In one embodiment, the effective concentration is about 1×10¹². In another embodiment, the effective concentration is about 1×10¹³.

The actual pharmaceutically effective amount or therapeutic dosage will of course depend on factors known by those skilled in the art such as age and weight of the patient, route of administration and severity of disease. In any case the rhabdovirus will be administered at dosages and in a manner which allows a pharmaceutically effective amount to be delivered based upon patient's unique condition.

Generally, for the treatment and/or alleviation of the diseases, disorders and conditions mentioned herein and depending on the specific disease, disorder or condition to be treated, the potency of the specific rhabdovirus, the specific route of administration and the specific pharmaceutical formulation or composition, the rhabdovirus will generally be administered for example, twice a week, weekly, or in monthly doses, but can significantly vary, especially, depending on the before-mentioned parameters. Thus, in some cases it may be sufficient to use less than the minimum dose given above, whereas in other cases the upper limit may have to be exceeded. When administering large amounts it may be advisable to divide them up into a number of smaller doses spread over the day.

It will be understood that the skilled artisan is able to choose and combine the different alternatives within the method or the process according to the invention without undue burden. For example, the skilled artisan can freely perform the optional process steps as needed and combine them in different setups, such as with different host cells, specific rhabdoviruses, or specific methods of clarifying the cell culture. In the following some preferred embodiments are described, however, the method and the process of the invention shall not be construed to be limited by these embodiments.

In one embodiment the method or the process according to the invention allows for preparation, production and/or purification of a vesiculovirus, preferably a vesicular stomatitis virus, more preferably a vesicular stomatitis virus, wherein the glycoprotein G of the vesicular stomatitis virus is replaced by the glycoprotein GP of Lymphocyte choriomeningitis virus (LCMV), in/from a cell culture, and comprises the following steps:
(i) obtaining a virus harvest from the cell culture by:
   a. adding directly to the cell culture a viral release agent, followed by clarifying the cell culture, preferably via depth filtration, tangential flow filtration or centrifugation, and recovery of the virus harvest in the supernatant,
   OR
   b. subjecting the cell culture to a filtration step, preferably depth filtration followed by rinsing of the filter, preferably the depth filter with a viral release agent, and recovery of the virus harvest in the supernatant.

In another embodiment the method or the process according to the invention allows for preparation, production and/or purification of a vesiculovirus, preferably a vesicular stomatitis virus, more preferably a vesicular stomatitis virus, wherein the glycoprotein G of the vesicular stomatitis virus is replaced by the glycoprotein GP of Lymphocyte choriomeningitis virus (LCMV), in/from a cell culture, and comprises the following steps:
(i) obtaining a virus harvest from the cell culture by:
   a. adding directly to the cell culture a viral release agent, wherein the viral release agent is a solid salt or an aqueous salt solution, followed by clarifying the cell culture, preferably via depth filtration, tangential flow filtration or centrifugation, and recovery of the virus harvest in the supernatant,
   OR
   b. subjecting the cell culture to a filtration step, preferably depth filtration followed by rinsing of the filter, preferably the depth filter with a viral release agent, wherein the viral release agent is an aqueous salt solution and recovery of the virus harvest in the supernatant.

In another embodiment the method or the process according to the invention allows for preparation, production and/or purification of a vesiculovirus, preferably a vesicular stomatitis virus, more preferably a vesicular stomatitis virus, wherein the glycoprotein G of the vesicular stomatitis virus is replaced by the glycoprotein GP of Lymphocyte choriomeningitis virus (LCMV), in/from a cell culture, and comprises the following steps:
(i) obtaining a virus harvest from the cell culture by:
   a. adding directly to the cell culture a viral release agent, wherein the viral release agent is a solid salt or an aqueous salt solution and the salt concentration in the cell culture is increased, followed by clarifying the cell culture, preferably via depth filtration, tangential flow filtration or centrifugation, and recovery of the virus harvest in the supernatant,
   OR
   b. subjecting the cell culture to a filtration step, preferably depth filtration followed by rinsing of the filter, preferably the depth filter with a viral release agent, wherein the viral release agent is an aqueous salt solution and recovery of the virus harvest in the supernatant.

In another embodiment the method or the process according to the invention allows for preparation, production and/or purification of a vesiculovirus, preferably a vesicular stomatitis virus, more preferably a vesicular stomatitis virus, wherein the glycoprotein G of the vesicular stomatitis virus is replaced by the glycoprotein GP of Lymphocyte choriomeningitis virus (LCMV), in/from a cell culture, and comprises the following steps:
(i) obtaining a virus harvest from the cell culture by:
   a. adding directly to the cell culture a viral release agent, wherein the viral release agent is a solid salt or an aqueous salt solution and the salt concentration in the cell culture is increased by at least approximately 0.01 M, 0.05 M, 0.1 M, 0.15 M, 0.2 M, 0.25 M, 0.3 M, 0.35 M, 0.4 M, 0.45 M or 0.5 M, followed by clarifying the cell culture, preferably via depth filtration, tangential flow filtration or centrifugation, and recovery of the virus harvest in the supernatant,
   OR
   b. subjecting the cell culture to a filtration step, preferably depth filtration followed by rinsing of the filter, preferably the depth filter with a viral release agent, wherein the viral release agent is an aqueous salt solution having a concentration of at least approximately 0.01 M, 0.05 M, 0.1 M, 0.15 M, 0.2 M, 0.25 M, 0.3 M, 0.35 M, 0.4 M, 0.45 M or 0.5 M, and recovery of the virus harvest in the supernatant.

In another embodiment the method or the process according to the invention allows for preparation, production and/or purification of a vesiculovirus, preferably a vesicular stomatitis virus, more preferably a vesicular stomatitis virus, wherein the glycoprotein G of the vesicular stomatitis virus is replaced by the glycoprotein GP of Lymphocyte choriomeningitis virus (LCMV), in/from a cell culture, and comprises the following steps:
(i) obtaining a virus harvest from the cell culture by:
   a. adding directly to the cell culture a viral release agent, wherein the viral release agent is a solid salt or an aqueous salt solution and the salt concentration in the cell culture is increased from about 0.01 M to about 5 M, from about 0.05 M to about 5 M, about 0.1 M to about 5 M, about 0.15 M to about 5 M, about 0.2 M to about 5 M, about 0.25 M to about 5 M, about 0.3 M to about 5 M, about 0.35 M to about 5 M, about 0.4 M to about 5 M, about 0.45 M to about 5 M, or about 0.5 M to about 5 M, followed by clarifying the cell culture, preferably via depth filtration, tangential flow filtration or centrifugation, and recovery of the virus harvest in the supernatant,
   OR
   b. subjecting the cell culture to a filtration step, preferably depth filtration followed by rinsing of the filter, preferably the depth filter with a viral release agent, wherein the viral release agent is an aqueous salt solution having a concentration from about 0.01 M to about 5 M, from about 0.05 M to about 5 M, about 0.1 M to about 5 M, about 0.15 M to about 5 M, about 0.2 M to about 5 M, about 0.25 M to about 5 M, about 0.3 M to about 5 M, about 0.35 M to about 5 M, about 0.4 M to about 5 M, about 0.45 M to about 5 M, or about 0.5 M to about 5 M, and recovery of the virus harvest in the supernatant.

In a further preferred embodiment, any of the aforementioned embodiments may further comprise the steps of:
(ii) (Optional) reducing the salt concentration of the harvest obtained after step (ia) or (ib), preferably by dilution, diafiltration or dialysis,
(iii) (Optional) treating the rhabdovirus harvest with a DNA degrading nuclease, preferably with benzonase or salt active nuclease,
(iv) capturing the rhabdovirus by loading the solution obtained after any of steps (i) to (iii) on a cation exchanger, preferably a monolith, a resin, or a membrane, more preferably a monolith adsorber,
(v) elution of the rhabdovirus and recovery of the eluate,
(vi) (Optional) polishing the rhabdovirus eluate of step (vii), preferably via size exclusion, multi modal size exclusion, ion exchange and/or tangential flow filtration,
(vii) (Optional) exchanging buffer of polished rhabdovirus eluate, preferably via ultrafiltration and diafiltration or dialysis,
(viii) (Optional) filtering sterilely of rhabdovirus.

In a further related embodiment, any of the aforementioned embodiments may further comprise the steps of:
(ii) (Optional) reducing the salt concentration of the harvest obtained after step (ia) or (ib), preferably by dilution, diafiltration or dialysis,
(iii) treating the rhabdovirus harvest with a DNA degrading nuclease, preferably with benzonase or salt active nuclease,
(iv) capturing the rhabdovirus by loading the solution obtained after any of steps (i) to (iii) on a cation exchanger, preferably a monolith, a resin, or a membrane, more preferably a monolith adsorber,
(v) elution of the rhabdovirus and recovery of the eluate,
(vi) (Optional) polishing the rhabdovirus eluate of step (vii), preferably via size exclusion, multi modal size exclusion, ion exchange and/or tangential flow filtration,
(vii) (Optional) exchanging buffer of polished rhabdovirus eluate, preferably via ultrafiltration and diafiltration or dialysis,
(viii) (Optional) filtering sterilely of rhabdovirus.

In a further related embodiment, any of the aforementioned embodiments may further comprise the steps of:
(ii) (Optional) reducing the salt concentration of the harvest obtained after step (ia) or (ib), preferably by dilution, diafiltration or dialysis,
(iii) treating the rhabdovirus harvest with a DNA degrading nuclease, preferably with benzonase or salt active nuclease,
(iv) capturing the rhabdovirus by loading the solution obtained after any of steps (i) to (iii) on a cation exchanger, preferably a monolith, a resin, or a membrane, more preferably a monolith adsorber,
(v) elution of the rhabdovirus and recovery of the eluate,
(vi) polishing the rhabdovirus eluate of step (vii), preferably via size exclusion, multi modal size exclusion, ion exchange and/or tangential flow filtration,
(vii) (Optional) exchanging buffer of polished rhabdovirus eluate, preferably via ultrafiltration and diafiltration or dialysis,
(viii) (Optional) filtering sterilely of rhabdovirus.

In a further related embodiment, any of the aforementioned embodiments may further comprise the steps of:
(ii) (Optional) reducing the salt concentration of the harvest obtained after step (ia) or (ib), preferably by dilution, diafiltration or dialysis,
(iii) treating the rhabdovirus harvest with a DNA degrading nuclease, preferably with benzonase or salt active nuclease,
(iv) capturing the rhabdovirus by loading the solution obtained after any of steps (i) to (iii) on a cation exchanger, preferably a monolith, a resin, or a membrane, more preferably a monolith adsorber,
(v) elution of the rhabdovirus and recovery of the eluate,
(vi) polishing the rhabdovirus eluate of step (vii), preferably via size exclusion, multi modal size exclusion, ion exchange and/or tangential flow filtration,
(vii) exchanging buffer of polished rhabdovirus eluate, preferably via ultrafiltration and diafiltration or dialysis,
(viii) (Optional) filtering sterilely of rhabdovirus.

In a further related embodiment, any of the aforementioned embodiments may further comprise the steps of:
(ii) (Optional) reducing the salt concentration of the harvest obtained after step (ia) or (ib), preferably by dilution, diafiltration or dialysis,
(iii) treating the rhabdovirus harvest with a DNA degrading nuclease, preferably with benzonase or salt active nuclease,
(iv) capturing the rhabdovirus by loading the solution obtained after any of steps (i) to (iii) on a cation exchanger, preferably a monolith, a resin, or a membrane, more preferably a monolith adsorber,
(v) elution of the rhabdovirus and recovery of the eluate,
(vi) polishing the rhabdovirus eluate of step (vii), preferably via size exclusion, multi modal size exclusion, ion exchange and/or tangential flow filtration,
(vii) exchanging buffer of polished rhabdovirus eluate, preferably via ultrafiltration and diafiltration or dialysis,
(viii) filtering sterilely of rhabdovirus.

In a further related embodiment, any of the aforementioned embodiments may further comprise the steps of:
(ii) reducing the salt concentration of the harvest obtained after step (ia) or (ib), preferably by dilution, diafiltration or dialysis,
(iii) treating the rhabdovirus harvest with a DNA degrading nuclease, preferably with benzonase or salt active nuclease,
(iv) capturing the rhabdovirus by loading the solution obtained after any of steps (i) to (iii) on a cation exchanger, preferably a monolith, a resin, or a membrane, more preferably a monolith adsorber,
(v) elution of the rhabdovirus and recovery of the eluate,
(vi) polishing the rhabdovirus eluate of step (vii), preferably via size exclusion, multi modal size exclusion, ion exchange and/or tangential flow filtration,
(vii) exchanging buffer of polished rhabdovirus eluate, preferably via ultrafiltration and diafiltration or dialysis,
(viii) filtering sterilely of rhabdovirus.

Further related to any of the foregoing specific embodiments it is preferred to prepare, produce or purify the vesiculovirus, preferably the vesicular stomatitis virus, more preferably the vesicular stomatitis virus, wherein the glycoprotein G of the vesicular stomatitis virus is replaced by the glycoprotein GP of Lymphocyte choriomeningitis virus (LCMV), in/from a cell culture comprising a mammalian host cell, preferably a mammalian host cell cultured in suspension and more preferably a HEK293 cell.

In a preferred embodiment relating to any of the foregoing specific embodiments, the RNA genome of the vesicular stomatitis virus consists of a coding sequence at least 98%, at least 99% or 100% identical to SEQ ID NO: 12.

### EXAMPLES

### Example 1: Drug Substance Upstream Manufacturing Process

Overview and Batch Definition

Based on one thawed HEK293 cell vial (UPS01), cells were expanded and cultivated in larger shake flask capacities (UPS02-UPS06). Following UPS06, a first bioreactor was seeded (UPS07) followed by cultivation and expansion of the cells into a second seed bioreactor (UPS08) and finally production bioreactor (UPS09). One batch of crude harvest containing the drug substance was produced in batch mode by infecting the cells 48 h or 56 h post seeding of the production bioreactor (UPS10). At time of infection, the cultivation temperature was either kept constant (process variant 1, 48 h) or was shifted from 37.0° C. to 34.0° C. (process variant 2, 56 h). Infection was performed with VSV-GP (wherein the glycoprotein G of the vesicular stomatitis virus is replaced with the glycoprotein GP of the lymphocytic choriomeningitis virus (LCMV) or VSV-GP-Cargo, i.e. a VSG-GP encoding for a further transgene, in particular a truncated CCL21 (1-79) protein or a CD80-Fc fusion protein. In the following, all processes were performed with VSV-GP as well as the different VSV-GP-Cargo variants (CCL21 (1-79) and CD80-Fc fusion) and for ease of reading will be referred with the single term VSV-GP(-Cargo). Harvest occurred 34±2 h after infection (UPS11). Subsequently, the entire working volume of approximately 200 L was subjected to downstream processing.

UPS01—Cell Thawing

HEK293 cells from one master cell bank (MCB) or working cell bank (WCB) vial were thawed in a heat block and adjusted to cultivation medium. After a centrifugation step, the resulting cell pellet was resuspended in medium and incubated in a 125 mL shake flask.

UPS02—UPS06—Cell Expansion in Shake Flasks

The HEK293 cells were propagated over five subcultivation stages in shake flasks. The next stage was started, when the inoculation criteria were reached. Here, the next higher shake flask capacity was used for each new subcultivation stage. If the maximum culture volume was exceeded, the remaining cells were discarded. For UPS07, as many 2000 mL shake flasks were used as needed/required to cover the inoculation criteria of the wave-mixed bioreactor.

UPS07—Cell Expansion in (First Seed) Bioreactor (Optional)

The last shake flask sub-cultivation (UPS06) was used to inoculate the first seed bioreactor for further cell expansion in batch mode. Wave bioreactor or stirred tank reactor are suitable for cell expansion. Prior to and during the inoculation, the pH value was controlled by addition of CO₂ via headspace to the bioreactor. During the entire cultivation, headspace aeration with air was present. The dissolved oxygen value was controlled by gassing air and oxygen using advanced controller in cascade regulation mode. The inoculum volume was calculated as well as the medium volume that had to be added depending on the viable cell concentration at the end of UPS06. The bioreactor was filled with the calculated medium volume. The medium was conditioned inside the bioreactor without aeration and pH control for at least 2 h. Just before inoculation, the aeration was started and the medium kept within the bioreactor with active pH control. The total conditioning time may not exceed 24 h. Once the conditions required for inoculation of the bioreactor were met, the cell suspension was transferred into the bioreactor via pumping. If the cell suspension is not entirely used during the inoculation, the remaining cells are discarded.

UPS08—Cell Expansion in Seed Bioreactor

A 50 L single use stirred-tank bioreactor was used for cultivation. Prior to and during the inoculation, the pH value was controlled by addition of CO₂ via sparger and headspace to the bioreactor. After inoculation, the low-side pH control by base addition was activated.

As alternative a glass bioreactor was used for cultivation. Prior to and during the inoculation, the pH value was controlled by addition of CO₂ via sparger and headspace to the bioreactor. After inoculation, the low-side pH control by base addition was activated. During the entire cultivation, headspace aeration with air was present. The DO percentage was controlled by submerse oxygen input and is regulated by an advanced controller.

For seeding, the viable cell concentration of the cell suspension contained in the wave-mixed bioreactor was determined. According to the required cell concentration in the bioreactor, the inoculum volume was calculated as well as the medium volume that had to be added. The bioreactor was filled with the calculated medium volume. The medium was conditioned inside the seed bioreactor without aeration and pH control for at least 2 h. At time of cell inoculation, the aeration was started and the medium kept within seed bioreactor with active pH control. The total conditioning time may not exceed 24 h. Once the conditions required for inoculation of the bioreactor were met, the cell suspension was transferred into the bioreactor via pumping. If the cell suspension is not entirely used during the inoculation, the remaining cells are discarded.

Anti-foam strategy (optional): To prevent the formation of foam during the cultivation, antifoam may be added to the culture using a time dependent pumping profile, which is started right after inoculation. Therefore, antifoam will be dosed directly to the cell suspension every six hours and the pump is running for one second each time. The added volume of antifoam is recorded by the process control system.

UPS09—Cell Expansion in Production Bioreactor

A 200 L single-use stirred-tank bioreactor was used for production. Prior to and during the inoculation, the pH value was controlled by addition of CO₂ via sparger and headspace. After inoculation, the low-side pH control by base addition was activated. During the entire cultivation, headspace aeration with air was present. The dissolved oxygen percentage will be controlled by submerse oxygen input and is regulated by a four-gas mixing module using advanced controller.

For seeding, the viable cell concentration of the cell suspension contained in the seed bioreactor was determined. According to the required cell concentration in the bioreactor, the inoculum volume was calculated as well as the medium volume that had to be added. The bioreactor was filled with the calculated medium volume. The medium was conditioned inside the production bioreactor without aeration and pH control for at least 2 h. Just before inoculation, the aeration was started and the medium kept within seed bioreactor with active pH control. The total conditioning time may not exceed 24 h. Once the conditions required for inoculation of the bioreactor were met, the cell suspension was transferred into the bioreactor via pumping. If the cell suspension is not entirely used during the inoculation, the remaining cells are discarded. To prevent the formation of foam during the cultivation, antifoam may be added to the culture using a time dependent pumping profile, which was started right after inoculation. Therefore, antifoam will be dosed directly to the cell suspension every six hours and the pump is running for one second each time. The added volume of antifoam was recorded by the process control system.

UPS10—Virus Infection Variant I

Infection with VSV-GP(-Cargo) was performed 48 h after cell inoculation of the production bioreactor stage, leading to a viable cell concentration between 1.0×10⁶ and 2.0×10⁶ cells/mL at time of infection. Virus was pre-diluted in conditioned cell culture medium. After the criteria were met for infecting the production bioreactor, control cells were seeded into two 250 mL shake flasks with cell suspension taken out of the production bioreactor. The shake flasks were incubated until the production bioreactor was harvested and the control cells were manipulated in the same manner as the production cells. At time of infection, the cultivation temperature of the production bioreactor will be constant in the range of 36.0° C. to 39.0° C.

UPS10—Virus Infection Variant II

Infection with VSV-GP(-Cargo) was performed 56 h after cell inoculation of the final reactor stage, leading to a viable cell concentration between 1.0×10⁶ and 2.0×10⁶ cells/mL at time of infection. Virus was pre-diluted in conditioned cell culture medium. After the criteria were met for infecting the production bioreactor, control cells were seeded into two 250 mL shake flasks with cell suspension taken out of the production bioreactor. The shake flasks were incubated until the production bioreactor was harvested and the control cells were manipulated in the same manner as the production cells. At time of infection, the cultivation temperature of the production bioreactor was shifted from 37.0° C. to 34.0° C. In case of excessive foam formation during the infection, antifoam may be added directly to the bioreactor to prevent clogging of e.g. aeration tubings. The added volume of antifoam was recorded by the process control system.

UPS11—Harvest

Harvest was conducted 34±2 h after infection. The harvesting procedure started by adding a 4 M to 5 M NaCl stock solution to the infected cell broth, leading to a concentration increase of approximately 0.2 M NaCl followed by an at least 10 min to 30 min incubation period before start of transfer and cell separation. All parameters were controlled as during cultivation unless otherwise stated. After a defined time of incubation, the temperature, DO and pH control were deactivated. Afterwards the infected cell broth was transferred for downstream processing.

Process Performance Data

Exemplary results from process monitoring and analytical in-process controls of VSV-GP-CCL21 (1-79) harvest manufactured with the described process are summarized below. Process performances between the different VSV-GP(-Cargo) variants (no cargo, CCL21 (1-79) or CD80-Fc fusion) were comparable.
maximum cell-specific growth rate of p=0.7 d⁻¹ in production bioreactor;
total cell concentration of 2-3×10⁶ cells per milliliter in harvest with viability of >90%;
infectious virus content in final cell culture supernatant of about 2×10⁹ TCID₅₀/mL, content of virus genomes in cell culture supernatant of about 3-3.5×10¹⁰ TCID₅₀/mL.

### Example 2: Drug Substance Downstream Manufacturing Process

Overview and Batch Definition

Manufacturing of VSV-GP-(Cargo) was based on a one batch production. All downstream unit operations were performed in one cycle without any split-up and pooling steps. The salt-treated harvest (UPS11) was clarified by depth filtration or tangential flow microfiltration, followed by a nuclease digestion step. The clarified harvest was further diluted before purification by monolithic cation exchange chromatography in bind-elute mode (DPS03). At this point a hold step was performed, where the intermediate was stored overnight. On the second downstream process day, the intermediate was purified by multimodal chromatography in flow-through mode (DPS04). The flow-through was collected and processed by an ultra-/diafiltration step (DPS05). The final filtration and formulation step (DPS06) resulted in a single batch, which was aliquoted into cell culture bottles, packaged and frozen at −80° C. (−86° C. to −70° C.).

Harvest Variant I—Depth Filtration

The depth filtration step was performed to clarify the bulk harvest. Therefore, VSV-GP(-Cargo) containing crude harvest was pumped from a STR® 200 bioreactor through a 3M™ Zeta Plus™ Encapsulated System depth filtration capsules into a single-use bag. Pressure maximum was 0.9 bar. Clarification was conducted at room temperature. The 3M™ Zeta Plus™ Encapsulated System depth filtration capsules were sanitized by 0.5 M—NaOH for disposal.

Harvest Variant II—Tangential Flow Microfiltration

The tangential flow microfiltration step was performed to clarify the bulk harvest. Therefore, the single use bioreactor was connected to the microfiltration module fitted with a 0.65 μm mPES hollow fibre membrane with an effective length of 41.5 cm and an inner fiber diameter of 0.75 mm. Filtration was performed at constant retentate flow rate with a shear rate of 2100 s⁻¹ and a maximal trans-membrane pressure of <0.7 bar. Microfiltration was performed until the inlet pressure increased to >0.7 bar. 2-5 liters of concentrated cell suspension were expected to remain in bioreactor vessel. Permeate was collected in a 500 L disposable bag. Temperature and pH were controlled to 37° C., pH 7.1 until the probes have no longer contact to the harvest.

DPS02—Enzymatic Digestion

Process step DPS02 is an enzymatic digest of the filtrate from DPS01, performed by SAN-HQ (ArcticZymes®) endonuclease. Inside a laminar flow at USP department, SAN-HQ endonuclease stock solution was equilibrated to room temperature and diluted in buffer (50 mM Tris, pH 7.5) in a separate single-use bag. Magnesium chloride was added as a co-factor for the enzyme. Digestion buffer was added successively to the intermediate (clarified harvest). Resulting intermediate was mixed for >10 min and incubated for >20 min at room temperature. After nuclease treatment, the intermediate was diluted 1:2 with 0.1 M Tris buffer to a defined pH- and conductivity value. The purpose of the enzymatic digestion step is the removal of nucleic acid (DNA and RNA) present in the suspension.

Bioburden Reduction Strategy at Clarified Harvest Level—Variant I

For reduction of a putative bioburden contamination during the harvest process, a bioburden reduction filtration using a 0.45 μm/0.2 μm Sartopore® 2 MaxiCaps® Size 1 (filters were connected in series) was conducted. The filter can be connected in series to the depth filter or at the permeate port of the microfiltration system. Filter was changed if the trans-membrane pressure was >0.9 bar. After filtration, the 0.45 μm/0.2 μm Sartopore® 2 MaxiCaps® Size 1 filter was removed from the assembly and tested for integrity.

Bioburden Reduction Strategy at Clarified Harvest Level—Variant II

For reduction of a putative bioburden contamination during the harvest process, a bioburden reduction filtration using a 5 μm polypropylene filter Kleenpak Nova 10″ Profile II 0.5 μm or 0.45 μm/0.2 μm Sartopore® 2 MaxiCaps® Size 1 was conducted. Filtration was performed after nuclease treatment and after dilution. Filter was changed if the trans-membrane pressure was >0.9 bar. After filtration, the filter was removed from the assembly and tested for integrity.

DPS03—Cation Exchange Chromatography

Process step DPS03 was a capture step performed with cation exchange chromatography on CIMmultus™ SO₃— monolith. The binding buffer contained 50 mM Tris, pH 7.5, optionally, supplemented with different Arginine concentrations. The chromatography was performed in bind/elute mode with step elution. VSV-GP(-Cargo) purification was performed at room temperature. The eluate was collected in one bag. Load was conducted at a flow rate of 5 or 10 CV/h. Monolith was washed with 10 CV or 50 CV and a volume flow of 5 CV/h or 10 CV/h. Elution was conducted at a flowrate of 120 CV/h or 10 CV/h. Collected eluate was stored at 2-8° C. overnight. The purpose of the step was the concentration of virus and removal of impurities (specifically residual host cell DNA and host cell proteins).

DPS04—Multimodal Size-Exclusion Chromatography

Process step DPS04 was a multimodal chromatography process step performed on Akta™ ready gradient with FteadyToProcess™ Capto® Core 700 with a bed high of 20 cm and a bed volume of 2.5 L. The chromatography was performed in flow-through mode. The resin was conditioned using elution buffer of cation exchange chromatography. Capture eluate incubated at least 30 min at room temperature before polishing step. VSV-GP(-Cargo) polishing was conducted at room temperature and at a constant velocity of 42 cm/h. The flow-through was collected in one bag. The purpose of the step was the final removal of minor impurities (specifically nuclease residues, HCP).

DPS05—Ultra/Diafiltration

A buffer exchange step was performed by a two-step diafiltration. Here the ultrafiltration module MiniKros® hollow fiber modules (Repligen) with a cutoff of 750 kD was used. In the first step, constant volume diafiltration was conducted, whereby the initial volume of intermediate was 6× diafiltrated against diafiltration buffer at a constant crossflow velocity which resulted in a maximum shear stress of 3000 s⁻¹. In step 2 the diafiltrated retentate was concentrated to a defined retentate volume at a constant crossflow velocity which resulted in a maximum shear stress of 3000 s⁻¹. The concentrated intermediate was drained into a 5 L single-use bag. The purpose of the step was a matrix exchange against the diafiltration buffer.

DPS06—Filling and Freezing

Diafiltration retentate was filtrated through a 0.45 μm/0.2 μm filter (Sartopore® MidiCaps®) and collected in a bag or bottle as suspension. The final primary packing material was connected aseptically to the bag/bottle filled by pumping. Then, the bottles were vacuum-sealed and packed in plastic foil. After that, the suspension was frozen and stored at −80° C. The purpose of the 0.2 μm filter was a final bioburden reduction. The freezing procedure allowed a storage condition of the drug substance suspension until further processing.

Process Performance Data

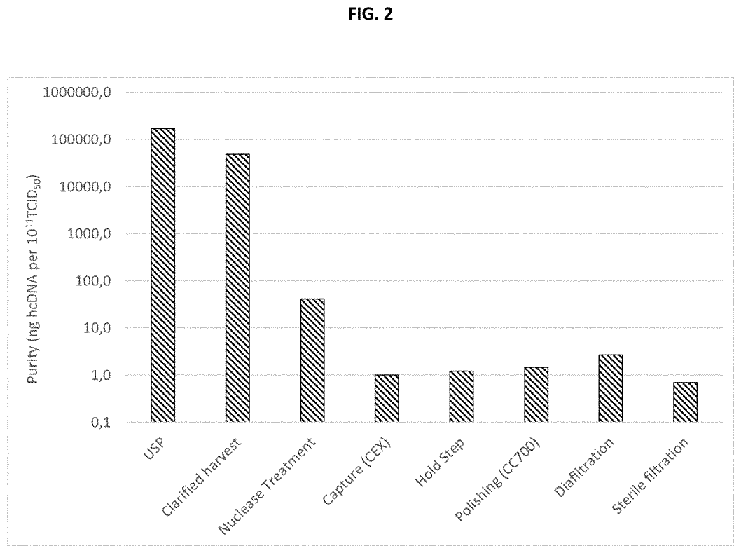

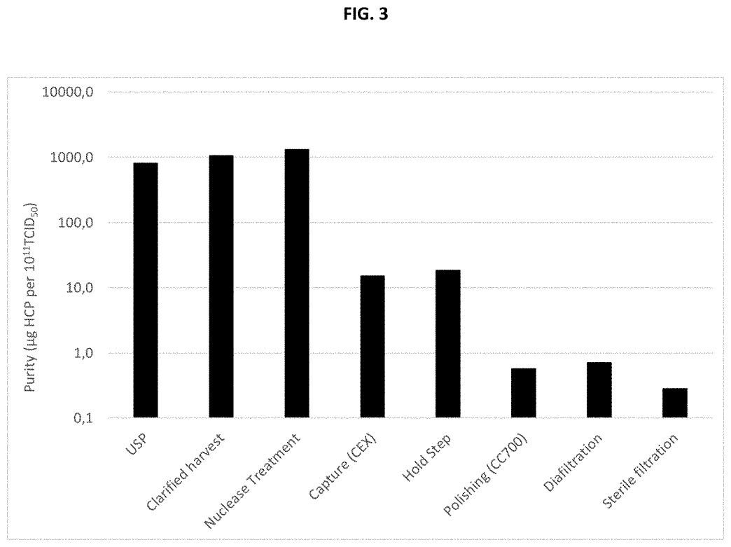

Exemplary results from process monitoring and analytical in-process controls of VSV-GP(-Cargo) harvest to drug substance manufactured with the described process using variant I in any process step are summarized in Table 1 and FIGS. 2 to 3. The impurity reduction match the high requirements and standards for an oncolytic virus drug product. The performance of impurity reduction from upstream to final drug substance corresponds to a log reduction value of ca. 3.5 for host cell protein (<0.4 μg/ml), and 5.4 for host cell DNA (final concentration <3 ng/ml), which is well below typical limits of generic impurity restrictions in biologicals development (e.g. 10 ng of hcDNA per dose according to W.H.O. guidelines). The overall yield allows for sufficient highly concentrated viral doses even for the highest demands.

TABLE 1
Example of infectious VSV-GP(-Cargo) content in manufacturing
unit operations, recovery calculated based on TCID₅₀ of initial
culture supernatant (centrifuged, salt treated).

| | | Virus content | Total |
|---|---|---|---|
| Process intermediate | Volume (L) | (TCID₅₀/mL) | recovery (%) |
| Culture supernatant | 198.32 | 1.49E+09 | 100% |
| Clarified harvest | 205.10 | 1.26E+09 | 87% |
| DNA Degradation | 206.25 | 9.67E+08 | 67% |
| Capture (CEX) | 0.84 | 2.41E+11 | 69% |
| Hold Step | 0.84 | 1.98E+11 | 56% |
| Polishing (CC700) | 1.36 | 1.10E+11 | 51% |
| Diafiltration | 1.22 | 4.90E+10 | 20% |
| Bioburden filtration | 1.69 | 4.28E+10 | 24% |

### Example 3: Testing of Different Additives Pre-Harvest

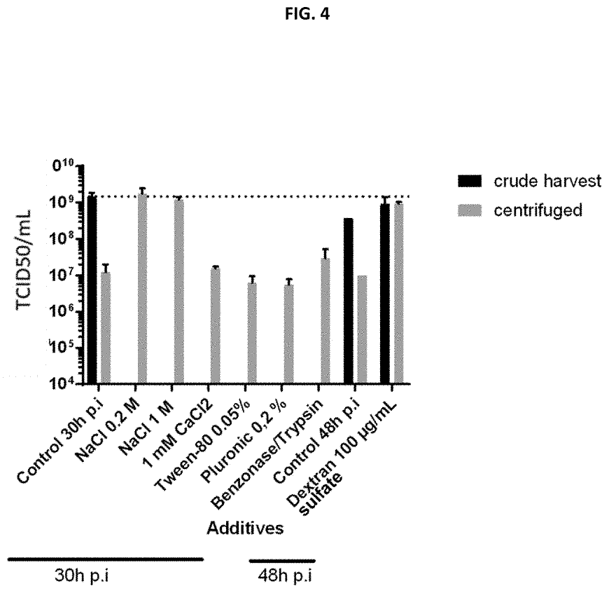

FIG. 4

Low harvest titers of VSV-GP were observed in the supernatant after a clarification by centrifugation when compared to non-centrifuged harvest material. It was hypothesized that the VSV-GP was interacting and binding with host cell membrane fragments. These fragments with bound VSV-GP would then pellet. Additives were proposed to disrupt the interaction between the membrane fragments allowing for pelleting of the membrane fragments and sufficient clarification of the VSV-GP that remained ‘free’ in the supernatant. Clarified harvest material was aliquoted into 15 mL fractions at a time point of 30 h post infection and additives added to final concentrations shown in FIG. 4. Samples containing NaCl, CaCl₂, Tween, Pluronic were incubated for 10 minutes at room temperature and centrifuged at 355 g for 3 minutes. Samples containing Benzonase and Trypsin were incubated for 30 minutes at 37° C. and centrifuged at 355 g for 3 minutes. Samples containing dextran sulfate were incubated for 18 h in culture conditions and centrifuged at 355 g for 3 minutes. The supernatant was then tested using TCID₅₀. The highest recoveries were observed for two concentrations of NaCl (0.2 M and 1 M) and 100 μg/mL dextran sulfate, indicating that these additives successfully released free virus particles into the supernatant. The other additives did not show a significant improvement in titer after centrifugation.

### Example 4: Further Testing of Different Additives and Concentrations

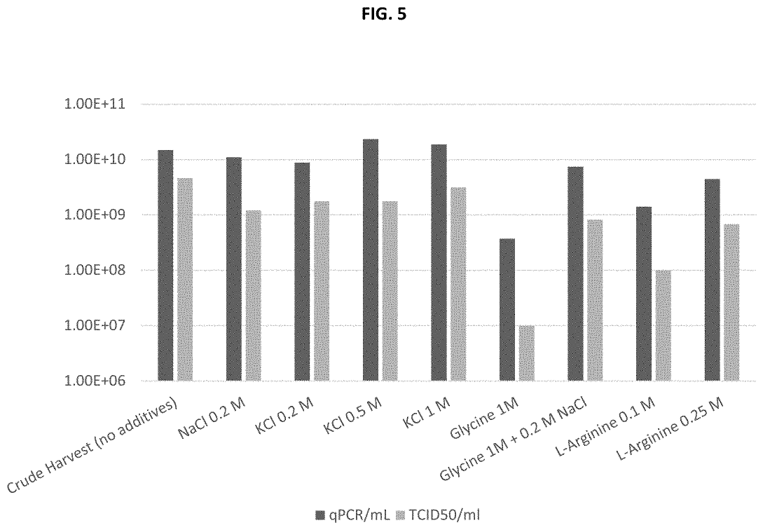

FIG. 5

Further additive testing was conducted to explore if the titer of VSV-GP at time of harvest could be improved with alternative additives. Additives were selected based on how they interacted with the virus; potassium chloride to test another monovalent ion, glycine which could increase the solubility and create exclusionary pressure, and L-Arginine which relaxes hydrophobic interactions and have 2+/1− charges at neutral pH. Clarified harvest material was aliquoted into 15 mL fractions at a time point of 30 h post infection and additives added to final concentrations shown, incubated for 10 minutes at room temperature and centrifuged at 355 g for 3 minutes. The supernatant was then tested using TCID₅₀ and qPCR. Non-treated crude harvest which had not been centrifuged showed high titers of infective VSV-GP, the interaction of VSV-GP with host cell debris did not inhibit VSV-GP's ability to infect new cells however large amount of debris is an impurity which is widely observed to negatively impact process performance of subsequent downstream processing steps. Of all the additives tested in FIG. 5, only KCl showed improved infective recovery over NaCl. It was also observed that increasing the concentration of KCl improved the VSV-GP release. It was decided however that as NaCl demonstrated good release of VSV-GP and is used for elution in the cation exchange capture, and would be used in other process steps, it would be used as an additive for VSV-GP harvest.

### Example 5: Testing of Minimum NaCl Concentration

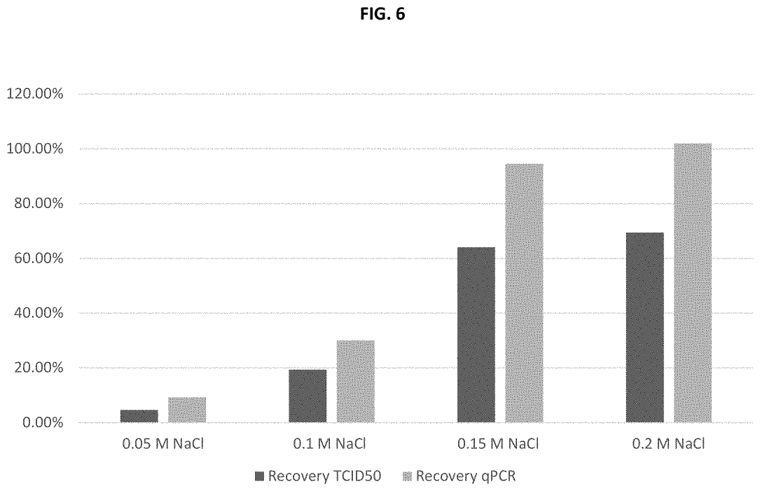

FIG. 6

NaCl was selected as the additive for release of VSV-GP at time of harvest because it demonstrated high infective recovery and would be present in different concentrations throughout the process. The study explored if lower concentrations of NaCl below 0.2 M could have comparable virus release effects because this would reduce the amount of dilution buffer required to reduce the conductivity of the feed before capture of VSV-G using cation exchange chromatography, a salt sensitive process step. Clarified harvest material was aliquoted into 15 mL fractions at a time point of 30 h post infection and NaCl added to final concentrations shown in FIG. 6, incubated for 10 minutes at room temperature and centrifuged at 355 g for 3 minutes. The supernatant was then tested using TCID₅₀. Infective recoveries using 0.2 M NaCl were shown to be the highest although lower concentrations also worked to a certain extent. This concentration thus appeared most promising for high recoveries of VSV-GP at time of harvest, whilst minimizing the dilution factor required to achieve capture of VSV-GP in the subsequent cation exchange step.

### Example 6: Exemplary Process Performance Data

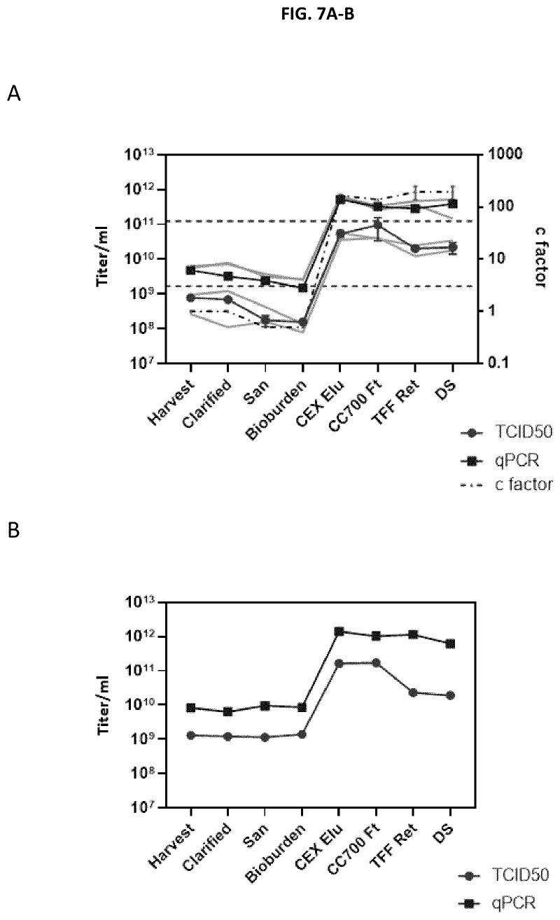

FIGS. 7A+B

Here, further exemplary performance data are shown for the process as described in detail in Examples 1 and 2 for infection of cell with VSV-GP and in culture volumes of 50 L (FIG. 7A) or 4 L (FIG. 7B). Briefly, cells were cultured in suspension for 48 h, VSV-GP was added at an MOI of 0.0005, virus was harvested and dissociated from host cell debris by addition of final concentration of 0.2 M NaCL at a TOH of 36 h. VSV-GP was clarified to remove host cell debris using 0.65 μm hollow fiber microfiltration and treated with SAN-HQ nuclease to reduce hcDNA. The feed was prepared before chromatographic capture by a 0.5 μm depth filtration and dilution 1:2 into binding buffer (final concentration 50 mM Tris, pH 7.5). The virus suspension was captured and concentrated using a cation exchange monolith, elution was achieved using a salt step. The capture material was held overnight, before undergoing chromatographic polishing using a multimodal size exclusion CC700 resin. This step further purified the VSV-GP from HCP and hcDNA not removed during capture. Finally the polished eluate was diafiltered into formulation buffer and sterile filtered using 0.2 μm filter.

### Example 7: Testing of MgCl₂ as Alternative Additive During Harvest

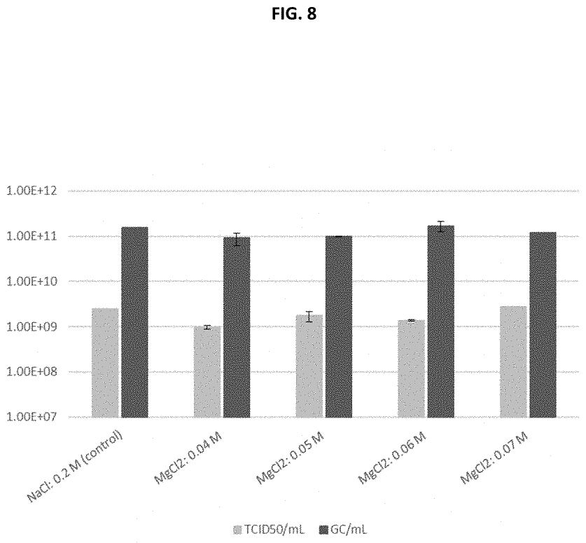

FIG. 8

In addition to previously tested excipients, magnesium chloride was added at time of harvest for release of VSV-GP(-Cargo) from infected cells. The effect of magnesium chloride as divalent cation was investigated, as it is highly soluble in water and shows similar ionic strength compared to sodium chloride at lower concentrations. Furthermore, magnesium cations (Mg²⁺) play an important role as cofactor during enzymatic digestion of nucleic acids for removal of free DNA/RNA. At time of harvest, the virus suspension was divided into 5- or 40-mL aliquots, incubated (10 min at 34° C.) with different amounts of magnesium chloride (add. 0.04 M to 0.07 M), as shown in FIG. 8 and centrifuged at 1000 g for 5 min. The supernatant was analyzed using TCID₅₀ and GC qPCR. A NaCl-treated sample (add. 0.2 M) was carried along as control sample (similar ionic strength compared to 0.07 M MgCl₂).

NaCl-treated crude harvest (control, add. 0.2 M) showed a similar virus titer (TCID₅₀ and GC qPCR) compared to MgCl₂-treated crude harvest (add. 0.07 M). Lower concentrations of MgCl₂ led to lower release of VSV-GP(-Cargo). Magnesium chloride represent a possible alternative for virus release from HEK293 cells during harvest. Also, the lower conductivity in the salt treated virus suspension reduces the amount of dilution buffer required to adjust the conductivity for the subsequent cation exchange chromatography step (capture).

### Example 8: Filter Flush after Depth Filtration for Virus Release Using NaCl

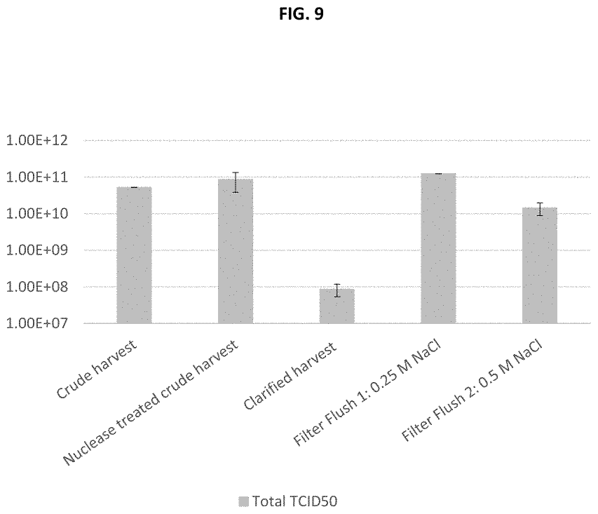

FIG. 9

Apart from the addition of sodium chloride for release of VSV-GP(-Cargo) at time of harvest, virus elution using a filter flush after depth filtration was tested. The study examined if VSV-GP(-Cargo) is released from HEK293 cells after retention in the filter material.

At time of harvest VSV-GP(-Cargo) was incubated with benzonase for 30 min at 37° C. in a shake incubator. Depth filtration (3M™ Zeta Plus™) for clarification of bulk harvest was executed with a defined volumetric filter load (200 L/m²), constant volumetric flux (200 LMH) and a pressure maximum of 1.5 bar (pre-filter). After depth filtration, a filter flush with one fifth (20%) of the filtered volume was carried out using tris buffer containing sodium chloride (0.25 M and 0.5 M NaCl).

FIG. 9 is an excellent example for the interaction of VSV-GP(-Cargo) with HEK293 cells when no additives were added at time of harvest, as the infectious virus titer in the clarified harvest is quite low. Virus release using a first filter flush with 0.25 M NaCl was already sufficient for a total infectious virus recovery. A second filter flush with 0.5 M NaCl did release some further virus but the first filter flush already nearly “eluted” most of the virus. The required amount of NaCl for virus release using a filter flush is comparable to the amount of NaCl added at the time of harvest (add. 0.2 M). Despite the good recovery of VSV-GP(-Cargo) using a filter flush after depth filtration, addition of NaCl at time of harvest convince with simplicity in the procedure.

### Example 9: Exemplary Method to Determine the TCID₅₀

Cells and Viruses:

BHK-21 cells (#603126 (C13), CLS) are cultured in 5% CO₂ and 37° C. Medium (GMEM #21710082, Thermo) is supplemented with 8.7% FCS and 4.3% Tryptose Phosphate Broth. BHK-21 cells are washed with PBS and detached from the cell culture flask by incubation with TrypLE™ Select Enzyme at 37° C. for 6-8 min. Cells are taken up in medium, counted using the Flex2 (nova biomedical) and seeded on 96-well plates.

TCID₅₀ Assay:

In 96-well plates 10⁴ BHK-21 cells in 100 μl supplemented GMEM are seeded per well. 24 h later, the adherent cells are infected with 11 0.5 log₁₀ serial dilutions of the virus or the diluent alone (negative control) before incubation for three days at 37° C., −5% CO₂. Brightfield images of the cell culture wells are taken with the Cytation5 Multi-Mode Imaging Reader (BioTek) using a 4× objective. Whether the imaged wells are CPE positive or negative is assessed by eye (i.e. visually). The final titer [TCID₅₀/mL] is calculated by the formula of Spearman and Kärber. For each virus sample infections with the serial dilutions are performed in a total of eight plates at the same day. Based on those eight replicates the TCID₅₀/mL is calculated as described above (single measurement).

1. A method of producing a rhabdovirus in a cell culture comprising the step of:
(i) obtaining a rhabdovirus harvest from the cell culture by:
a. adding directly to the cell culture a viral release agent, followed by clarifying the cell culture and recovery of the rhabdovirus harvest in the supernatant,
OR
b. subjecting the cell culture to a filtration step followed by rinsing of the filter with a viral release agent, and recovery of the rhabdovirus harvest in the supernatant.

2. The method according to claim 1, wherein the rhabdovirus is a vesiculovirus.

3. The method according to claim 2, wherein the vesiculovirus is a vesicular stomatitis virus.

4. The method according to claim 3, wherein the glycoprotein G of the vesicular stomatitis virus is replaced by the glycoprotein GP of Lymphocyte choriomeningitis virus (LCMV).

5. The method according to claim 1, wherein the viral release agent in step (ia) is a solid salt or an aqueous salt solution, and the viral release agent in step (ib) is an aqueous salt solution.

6. The method according to claim 5, wherein in step (ia) the salt concentration in the cell culture is increased by at least approximately 0.01 M, 0.05 M, 0.1 M, 0.15 M, 0.2 M, 0.25 M, 0.3 M, 0.35 M, 0.4 M, 0.45 M or 0.5 M and the aqueous salt solution in step (ib) has a concentration of at least approximately 0.01 M, 0.05 M, 0.1 M, 0.15 M, 0.2 M, 0.25 M, 0.3 M, 0.35 M, 0.4 M, 0.45 M or 0.5 M.

7. The method according to claim 5, wherein in step (ia) the increase in salt concentration in the cell culture and in step (ib) the concentration of the aqueous salt solution is from about 0.01 M to about 5 M, 0.05 M to about 5 M, about 0.1 M to about 5 M, about 0.15 M to about 5 M, about 0.2 M to about 5 M, about 0.25 M to about 5 M, about 0.3 M to about 5 M, about 0.35 M to about 5 M, about 0.4 M to about 5 M, about 0.45 M to about 5 M, or about 0.5 M to about 5 M.

8. The method according to claim 5 wherein the salt is NaCl, KCl, MgCl₂, CaCl₂), NH₄Cl, NH₄ sulfate, NH₄ acetate or NH₄ bicarbonate.

9. The method according to claim 1, wherein the viral release agent is an amino acid.

10. The method according to claim 1, wherein the viral release agent is an acidic or basic amino acid.

11. The method according to claim 1, wherein the viral release agent is arginine.

12. The method according to claim 1, wherein the viral release agent is a sulfated polysaccharide.

13. The method according to claim 1, wherein the viral release agent is dextran sulfate.

14. The method according to claim 1, wherein in step (ia) by adding the viral release agent to the cell culture the ionic strength of the cell culture is increased.

15. The method according to claim 1, wherein in step (ia) by adding the viral release agent to the cell culture the ionic strength of the cell culture is increased by at least approximately 0.01 M, or at least approximately 0.05 M, or at least approximately 0.1 M, or at least approximately 0.15 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 5 M, or from about 0.05 M to about 5 M, or from about 0.1 M to about 5 M, or from about 0.15 M to about 5 M, or from about 0.2 M to about 5 M; and in step (ib) rinsing the filter with a viral release agent containing aqueous solution having a ionic strength of at least approximately 0.01 M, at least approximately 0.05 M, or at least approximately 0.1 M, or at least approximately 0.15 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 5 M, or from about 0.05 M to about 5 M, or from about 0.1 M to about 5 M, or from about 0.15 M to about 5 M, or from about 0.2 M to about 5 M.

16. The method according to claim 1, wherein the rhabdovirus is produced in a mammalian host cell.

17. The method according to claim 1, wherein the rhabdovirus is produced in a HEK293 cell.

18. The method according to claim 16, wherein the mammalian host cell is cultured in suspension.

19. The method of producing a rhabdovirus in a cell culture according to claim 1, further comprising the steps of:
(ii) reducing the salt concentration of the harvest obtained after step (ia) or (ib),
(iii) treating the rhabdovirus harvest with a DNA degrading nuclease,
(iv) capturing the rhabdovirus by loading the solution obtained after any of steps (i) to (iii) on a cation exchanger,
(v) elution of the rhabdovirus and recovery of the eluate,
(vi) polishing the rhabdovirus eluate of step (vii),
(vii) exchanging buffer of polished rhabdovirus eluate, and
(viii) filtering sterilely of rhabdovirus.

20. The method according to claim 19, wherein the cation exchanger is a monolith, a resin, or a membrane.

21. The method according to claim 20, wherein the cation exchanger is a monolith adsorber.

22. The method according to claim 1, wherein the rhabdovirus is formulated into a pharmaceutical composition.

23. A process for purifying a rhabdovirus from a cell culture infected with the rhabdovirus, comprising the step of:
(i) obtaining a rhabdovirus harvest from the cell culture by:
a. adding directly to the cell culture a viral release agent, followed by clarifying the cell culture, and recovery of the rhabdovirus harvest in the supernatant,
OR
b. subjecting the cell culture to a filtration step followed by rinsing of the filter with a viral release agent, and recovery of the rhabdovirus harvest in the supernatant.

24. The process according to claim 23, wherein the rhabdovirus is a vesiculovirus.

25. The process according to claim 23, wherein the rhabdovirus is a vesicular stomatitis virus.

26. The process according to claim 25, wherein the glycoprotein G of the vesicular stomatitis virus is replaced by the glycoprotein GP of Lymphocyte choriomeningitis virus (LCMV).

27. The process according to claim 23, wherein the viral release agent in step (ia) is a solid salt or an aqueous salt solution, and the viral release agent in step (ib) is an aqueous salt solution.

28. The process according to claim 27, wherein in step (ia) the salt concentration in the cell culture is increased by at least approximately 0.01 M, 0.05 M, 0.1 M, 0.15 M, 0.2 M, 0.25 M, 0.3 M, 0.35 M, 0.4 M, 0.45 M or 0.5 M and the aqueous salt solution in step (ib) has a concentration of at least approximately 0.01 M, 0.05 M, 0.1 M, 0.15 M, 0.2 M, 0.25 M, 0.3 M, 0.35 M, 0.4 M, 0.45 M or 0.5 M.

29. The process according to claim 28, wherein in step (ia) the increase in salt concentration in the cell culture and in step (ib) the concentration of the aqueous salt solution is from about 0.01 M to about 5 M, about 0.05 M to about 5 M, about 0.1 M to about 5 M, about 0.15 M to about 5 M, about 0.2 M to about 5 M, about 0.25 M to about 5 M, about 0.3 M to about 5 M, about 0.35 M to about 5 M, about 0.4 M to about 5 M, about 0.45 M to about 5 M, or about 0.5 M to about 5 M.

30. The process according to claim 27 wherein the salt is NaCl, KCl, MgCl₂, CaCl₂), NH₄Cl, NH₄ sulfate, NH₄ acetate or NH₄ bicarbonate.

31. The method according to claim 23, wherein the viral release agent is an amino acid.

32. The method according to claim 23, wherein the viral release agent is a polar, acidic or basic amino acid.

33. The method according to claim 23, wherein the viral release agent is arginine.

34. The process according to claim 23, wherein the viral release agent is a sulfated polysaccharide.

35. The process according to claim 23, wherein the viral release agent is dextran sulfate.

36. The method according to claim 23, wherein in step (ia) by adding the viral release agent to the cell culture the ionic strength of the cell culture is increased.

37. The method according to claim 23, wherein in step (ia) by adding the viral release agent to the cell culture the ionic strength of the cell culture is increased by at least approximately 0.01 M, or at least approximately 0.05 M, or at least approximately 0.1 M, or at least approximately 0.15 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 5 M, or from about 0.05 M to about 5 M, or from about 0.1 M to about 5 M, or from about 0.15 M to about 5 M, or from about 0.2 M to about 5 M; and in step (ib) rinsing the filter with a viral release agent containing aqueous solution having a ionic strength of at least approximately 0.01 M, or at least approximately 0.05 M, or at least approximately 0.1 M, or at least approximately 0.15 M, or at least approximately 0.2 M, or at least approximately 0.25 M, or at least approximately 0.3 M, or at least approximately 0.35 M, or at least approximately 0.4 M, or at least approximately 0.45 M, or at least approximately 0.5 M, or from about 0.01 M to about 5 M, or from about 0.05 M to about 5 M, or from about 0.1 M to about 5 M, or from about 0.15 M to about 5 M, or from about 0.2 M to about 5 M.

38. The process according to claim 23, wherein the rhabdovirus is purified from a mammalian host cell.

39. The process according to claim 23, wherein the rhabdovirus is purified from a HEK293 cell.

40. The process according to claim 38, wherein the mammalian host cell is cultured in suspension.

41. A process for purifying a rhabdovirus according to claim 17, further comprising the steps of:
(ii) reducing the salt concentration of the harvest obtained after step (ia) or (ib),
(iii) treating the rhabdovirus harvest with a DNA degrading nuclease,
(iv) capturing the rhabdovirus by loading the solution obtained after any of steps (i) to (iii) on a cation exchanger,
(v) elution of the rhabdovirus and recovery of the eluate,
(vi) polishing the rhabdovirus eluate of step (vii),
(vii) exchanging buffer of polished rhabdovirus eluate, and
(viii) filtering sterilely of rhabdovirus.

42. The process according to claim 41, wherein the cation exchanger is a monolith, a resin, or a membrane.

43. The process according to claim 42, wherein the cation exchanger is a monolith adsorber.

44. The process according to claim 23, wherein the rhabdovirus is formulated into a pharmaceutical composition.

45. A vesicular stomatitis virus, wherein the glycoprotein G of the vesicular stomatitis virus is replaced by the glycoprotein GP of Lymphocyte choriomeningitis virus (LCMV), produced or purified according to the method of claim 1.

46. The vesicular stomatitis virus produced or purified according to claim 45, wherein the RNA genome of the vesicular stomatitis virus consists of a coding sequence at least 98%, at least 99% or 100% identical to SEQ ID NO: 12.

47. The vesicular stomatitis virus produced or purified according to claim 45, wherein the amount of infectious particles is at least approximately about 1×10⁹, 2×10⁹, 3×10⁹, 4×10⁹, 5×10⁹, 6×10⁹, 7×10⁹, 8×10⁹, 9×10⁹, or at least approximately about 1×10¹⁰ as measured by TCID₅₀/mL.
